(19) United States
(12) Patent Application Publication
(10) Pub. No.: US 2012/0269822 A1
(43) Pub. Date: Oct. 25, 2012

(54) Anti-Botulinum Neurotoxin a Single Domain Antibody Antibodies

(76) Inventors: James D. Marks, Kensington, CA (US); Jianbo Dong, San Francisco, CA (US); Jianlong Lou, San Bruno, CA (US)

(21) Appl. No.: 13/502,711

(22) PCT Filed: Oct. 19, 2010
(86) PCT No.: PCT/US10/53271

§ 371 Date: Jul. 2, 2012

Related U.S. Application Data
(60) Provisional application No. 61/253,449, filed on Oct. 20, 2009.

(51) Int. Cl. C07K 16/12; C12N 15/13; A61K 39/40; A61P 25/00; C12N 5/10; C12N 1/21

(52) U.S. Cl. 424/167.1; 435/338; 435/252.33; 435/252.31; 435/252.3; 530/389.5; 536/23.53

(57) ABSTRACT

Antibodies that bind to botulinum neurotoxin(s) are disclosed herein, as well as related compositions and methods of use. The present disclosure provides antibodies that specifically bind a Botulinum neurotoxin (BoNT) and inhibit the activity of BoNT in cleavage of its substrate.

### CROSS-REFERENCE TO RELATED APPLICATIONS

This application claims priority benefit to U.S. provisional application Ser. No. 61/253,449 filed on Oct. 20, 2009, which application is incorporated herein by reference in its entirety.

### STATEMENT AS TO FEDERALLY SPONSORED RESEARCH

This invention was partially supported by a Defense Threat Reduction Agency contract 1-07-C-0030, National Institutes of Health cooperative agreement U01 AI056493 (J.D.M.). The government has certain rights in this invention.

### INTRODUCTION

Botulism is caused by botulinum neurotoxin secreted by members of the genus Clostridium and is characterized by flaccid paralysis, which if not immediately fatal requires prolonged hospitalization in an intensive care unit and mechanical ventilation. Naturally occurring botulism is found in infants or adults whose gastrointestinal tracts become colonized by Clostridial bacteria (infant or intestinal botulism), after ingestion of contaminated food products (food botulism), or in anaerobic wound infections (wound botulism). Botulinum neurotoxins (BoNTs) are also classified by the Centers for Disease Control (CDC) as one of the six highest-risk threat agents for bioterrorism (the “Category A agents”), due to their extreme potency and lethality, ease of production and transport, and need for prolonged intensive care.

### SUMMARY

Antibodies that bind to botulinum neurotoxin(s) are disclosed herein, as well as related compositions and methods of use. The present disclosure provides antibodies that specifically bind a Botulinum neurotoxin (BoNT) and inhibit the activity of BoNT in cleavage of its substrate.

The disclosure provides antibodies that specifically bind an alpha-exosite of a BoNT light chain (Lc) and inhibits its cleavage of its substrate. The disclosure provides antibodies that bind an alpha-exosite of a Botulinum neurotoxin A (BoNT/A) light chain (Lc) and inhibits cleavage of SNAP25 by the BoNT/A.

Antibodies provided by the present disclosure include heavy-chain only antibodies, and antigen binding fragments thereof, that contain at least one, two or all three heavy chain (V_{H}) complementarity determining region(s) (CDR(s)) of an antibody from clone Aa1, A26, A3, A16, A23, A10, Aa12, Aa6, Aa9, A8, A21, A19, Aa8, Aa5, Aa11, A8.1a, B01, B04, B12, B22, Bc1, Bc2, Bc3, Bc4, Bc5, Bc6, Bc7, Bc8, Bc9, Bc10, Bc11, Bc12, Bc13, or Bc14. Antibodies that contain at least a CDR1 of the VH of the antibody from clone Aa1 are provided.

The antibody may contain all V_{H} CDRs of an antibody from clone Aa1, A26, A3, A16, A23, A10, Aa12, Aa6, Aa9, A8, A21, A19, Aa8, Aa5, Aa11, A8.1a, B01, B04, B12, B22, Bc1, Bc2, Bc3, Bc4, Bc5, Bc6, Bc7, Bc8, Bc9, Bc10, Bc11, Bc12, Bc13, or Bc14.

The antibody may contain full-length V_{H} chain of an antibody from clone Aa1, A26, A3, A16, A23, A10, Aa12, Aa6, Aa9, A8, A21, A19, Aa8, Aa5, Aa11, B01, B04, B12, B22, Bc1, Bc2, Bc3, Bc4, Bc5, Bc6, Bc7, Bc8, Bc9, Bc10, Bc11, Bc12, Bc13, or Bc14.

The antibody may be a V_{H}H, Fab, (Fab′)₂, or other antigen-binding fragment of a V_{H}H.

The antibody may competititively bind to an epitope (e.g. α-exosite) on BoNT/A with an antibody from clone Aa1, A26, A3, A16, A23, A10, Aa12, Aa6, Aa9, A8, A21, A19, Aa8, Aa5, A8.1a, or Aa11. The antibody may competititively bind to an epitope on BoNT/B with an antibody from clone B01, B04, B12, B22, Bc1, Bc2, Bc3, Bc4, Bc5, Bc6, Bc7, Bc8, Bc9, Bc10, Bc11, Bc12, Bc13, or Bc14. The antibody may also be in a pharmaceutically acceptable excipient (e.g., in a unit dosage formulation).

Antibodies are provided herein that at least partially inhibit the catalytic activity (e.g. cleavage of its substrate) of a BoNT (e.g. BoNT/A L_{c} (light chain)). Such antibodies find use in methods of treating a subject exposed to a botulinum neurotoxin, where the methods can involve administering an effective amount of such an inhibitory anti-BoNT antibody to the subject so as to provide for inhibition of activity of botulinum neurotoxin in the subject. Such methods include treatment of a subject that suffers from intoxication by botulinum neurotoxin. Methods of the present disclosure include those that provide for administering an anti-BoNT antibody as disclosed herein in an amount effective to reverse BoNT-induced paralysis in a subject.

Nucleic acids provided herein encode one or more antibodies that are described herein. Cells containing such antibodies are also provided herein. Kits provided for inhibiting the cleavage activity of a Botulinum neurotoxin may include a composition containing one or more antibodies as described herein. The kits optionally also include instructional materials teaching the use of the composition to inhibit catalytic activity of a Botulinum neurotoxin.

### BRIEF DESCRIPTION OF THE DRAWINGS

FIG. 1. Selection of yeast displayed V_{H}H by using flow cytometry. Dot-plots of flow cytometry sorting of V_{H}H displaying yeast labeled with BoNT/A Lc are shown. For each of the three rounds of sorting, the concentration of BoNT/A Lc used to stain yeast is indicated. BoNT/A Lc binding is indicated on the Y-axis and the V_{H}H display level on the X-axis. The sort gates used for yeast collection are indicated and the yeast in these gates are colored green.

FIG. 2. SDS-PAGE analysis of V_{H}H (camelid heavy chain variable region derived from heavy chain only antibody) inhibition of GST-SNAP cleavage by BoNT/A Lc. (A) Inhibitory effect of 15 unique V_{H}H fragments. Each V_{H}H was incubated in a 50 fold molar excess over BoNT/A Lc for 3 min followed by the addition of GST-SNAP25. After 10 min of incubation, SNAP25 cleavage was analyzed by SDS-PAGE. (B) Effect of molar ratio on GST-SNAP25 cleavage. Two inhibitory and one non-inhibitory V_{H}H were incubated with varying fold molar excesses over BoNT/A Lc (1:1 to 70:1) for 30 min followed by the addition of GST-SNAP25. After 10 min of incubation, SNAP25 cleavage was analyzed by SDS-PAGE.

FIG. 3. Characterization of the Aa1 V_{H}H fragment. (A) Solution K_{D}. The solution K_{D} of the purified Aa1 V_{H}H fragment was measured by flow fluorimetry in a KinExA instrument. (B) Aa1 V_{H}H fragment IC₅₀ for SNAP25 cleavage by BoNT/A Lc. The indicated Aa1 V_{H}H concentration was incubated with BoNT/A Lc and the FRET substrate YsCsY and the initial rate of cleavage determined from the change in the YFP fluorescence reading. IC₅₀ was determined by fitting the initial rate and log Aa1 concentration to a sigmoidal dose-response (variable slope) model. (C) SDS-PAGE analysis of the impact of reducing agents on Aa1 V_{H}H inhibition of GST-SNAP cleavage by BoNT/A Lc. The Aa1 V_{H}H was incubated with no reducing agent (A), 20 mM glutathione reduced (B), or 14 mM mercaptaethanol (C) for 15 min at 37° C. followed by addition of BoNT/A Lc and GST-SNAP25. After 15 min, cleavage was analyzed by SDS-PAGE.

FIG. 4. Thermal denaturation and refolding of Aa1 V_{H}H. (A) Far UV CD spectra of Aa1 V_{H}H obtained at 10° C. (♦) before melting, 90° C. (▾) after melting, and 10° C. (▴) following the melting and refolding of the protein. (B) (C) Thermal denaturation (◯) and refolding (□) data of Aa1 V_{H}H obtained by CD spectroscopy at a wavelength of 216 nm (panel B) and 224 nm (panel C).

FIG. 5. Structure of the BoNT/A Lc endopeptidase/Aa1 V_{H}H complex. (A) BoNT/A Lc endopeptidase in gray complexed with the V_{H}H fragment in yellow with the CDR1, CDR2, and CDR3 regions colored blue, red, and green, respectively. The catalytic zinc is depicted as a red sphere is all figures. (B) Surface representation of the BoNT/A Lc highlighting the Aa1 V_{H}H binding site. Six hydrogen bonds between the endopeptidase and the V_{H}H fragment are indicated with yellow dashes. (C) The SNAP25 natural substrate colored in magenta from PDB code 1E1H superimposed onto the BoNT/A Lc/V_{H}H complex. The α-helical portion of SNAP25 that binds to the BoNT/A Lc α-exosite coincides with the alpha-helical tips of CDR1 and CDR3. (D) The same superposition from panel (C) highlighting the amino acid conservation between the SNAP25 α-exosite binding region and the Aa1 V_{H}H fragment. (E) The “belt” from the BoNT holostructure colored orange (from PDB code 3BTA) superimposed onto the BoNT/A Lc/V_{H}H complex. The α-helical tips of CDR1 and CDR3 coincide with an α-helical portion of the “belt” in a fashion similar to the SNAP25/V_{H}H supperposition shown in panel (C).

FIG. 6. Structural Comparison between Aa1 V_{H}H and the available V_{H}H structures in the PDB databank. (A) Structural alignment of the available V_{H}H fragment structures in the PDB colored gray with Aa1 V_{H}H colored yellow with the CDRs 1, 2, 3, colored blue, red and green, respectively. The unique CDR1 of Aa1 V_{H}H forms an extended loop with a small α-helix at the tip. All structural alignments were performed using the combinatorial extension (CE) method (73) and the PDB codes are listed in the amino acid sequence alignment of Supplemental Figure S2. (B) 180° rotation (along the y-axis) of the superposition shown in panel (A). (C) CE structural alignment of the V_{H}H fragment from PDB code 1F2X colored gray and Aa1 V_{H}H colored the same as in panels (A) and (B) with an RMSD of 2.0 Å.

FIG. 7. Panel A shows deduced protein sequences of BoNT/A binders (V_{H}H): Aa1, (SEQ ID NO:1), A26, (SEQ ID NO:2), A3 (SEQ ID NO:3), A16 (SEQ ID NO:4), A23 (SEQ ID NO:5), A10 (SEQ ID NO:6), Aa12 (SEQ ID NO:7), Aa6 (SEQ ID NO:8), Aa9 (SEQ ID NO:9), A8 (SEQ ID NO:10), A21 (SEQ ID NO:11), A19 (SEQ ID NO:12), Aa8 (SEQ ID NO:13), Aa5 (SEQ ID NO:14), Aa11 (SEQ ID NO:15), A8.1a (SEQ ID NO:16). Panel B shows protein sequences of BoNT/B binders: B01(SEQ ID NO:17), B04 (SEQ ID NO:18), B12 (SEQ ID NO:19), B22 (SEQ ID NO:20), Bc1 (SEQ ID NO:21), Bc2 (SEQ ID NO:22), Bc3 (SEQ ID NO:23), Bc4 (SEQ ID NO:24), Bc5 (SEQ ID NO:25), Bc6 (SEQ ID NO:26), Bc7 (SEQ ID NO:27), Bc8 (SEQ ID NO:28), Bc9 (SEQ ID NO:29), Bc10 (SEQ ID NO:30), Bc11 (SEQ ID NO:31), Bc12 (SEQ ID NO:32), Bc13 (SEQ ID NO:33), or Bc14 (SEQ ID NO:34).

FIG. 8 Thermal denaturation and refolding of Aa1 V_{H}H in the presence of 1 mM TCEP. (A) Far UV CD spectra of Aa1 V_{H}H with 1 mM TCEP obtained at 10° C. ( [symbol] ) before melting, and 10° C. (▪) following melting and “refolding” of the protein. (B) Thermal denaturation ( [symbol] ) and “refolding” (▪) of Aa1 V_{H}H in the presence of 1 mM TCEP obtained by CD spectroscopy at a wavelength of 216 nm. These data indicate that the Aa1 V_{H}H protein does not properly refold in the presence of 1 mM TCEP.

FIG. 9 Amino Acid Sequence Alignment of V_{H}H domains with Structures in the PDB databank. Analysis performed with Vector NTI using default amino acid letter coloring where identical residues are red on a yellow background, regions of high sequence conservation are dark blue on a light blue background, moderate blocks of similarity are black on a green background, weakly similar residues are dark green on a white background, and residues that are not similar are black on a white background. Each V_{H}H primary sequence is referenced according to its PDB code and chain ID. The complementarity determining regions (CDRs) are highlighted above the corresponding region, and the conserved immunoglobulin disulfide bond is indicated with a dashed line.

### DEFINITIONS

The following abbreviations may be used herein: BoNT, botulinum neurotoxin; BoNT/A, botulinum neurotoxin serotype A; BoNT/A Lc, botulinum neurotoxin serotype A light chain; BoNT/A Lc₄₂₅, truncated BoNT/A Lc containing residues 1-425; BoNT/A Lc₄₄₈, truncated BoNT/A Lc containing residues 1-448; CD, circular dichroism; CDR, complementarity determining region; Fab, antigen binding fragment of immunoglobulin with variable domain and first constant domain; FACS, fluorescent activated cell sorting; FRET, fluorescence resonance energy transfer; Hc, the C-terminal portion of the botulinum neurotoxin heavy chain; Hn, the N-terminal portion of the botulinum neurotoxin heavy chain; IC₅₀, 50% inhibitory concentration; IgG, immunoglobulin G; IPTG, isopropyl-β-D-thiogalactopyranoside; IMAC, immobilized metal affinity chromatography; K_{D}, dissociation equilibrium constant; kₒₙ, association rate constant; k_{off}, dissociation rate constant; mAb, monoclonal antibody; MFI, mean fluorescent intensity; PBS, phosphate buffered saline; PCR, polymerase chain reaction; scFv, single chain format of antibody variable regions; SD-CAA, selective growth dextrose casamino acids media; SG-CAA media, selective growth galactose casamino acids media; SNARE, Soluble N-ethylmaleimide-sensitive factor attachment protein receptor; SNAP25, synaptosome-associated protein of 25,000 daltons; VH, heavy chain variable region; V_{H}H, camelid heavy chain variable region derived from heavy chain only antibody.

A “BoNT polypeptide” refers to a Botulinum neurotoxin polypeptide (e.g., a BoNT/A polypeptide, a BoNT/B polypeptide, a BoNT/C polypeptide, and so forth). The BoNT polypeptide can refer to a full-length polypeptide or to a fragment thereof. Thus, for example, the term “BoNT/A polypeptide” refers to either a full-length BoNT/A (a neurotoxin produced by Clostridium botulinum of the type A serotype) or a fragment thereof (e.g. the Hc fragment). The Hc fragment of BoNT/A is an approximately 50 kDa C-terminal fragment (residues 873-1296) of BoNT/A (Lacy and Stevens (1999) J. Mol. Biol., 291: 1091-1104).

A “BoNT” serotype refers to one of the standard known BoNT serotypes (e.g. BoNT/A, BoNT/B, BoNT/C, BoNT/D, BoNT/E, BoNT/F, BoNT/G, etc.). BoNT serotypes differ from each other by as little as about 35% at the amino acid level (e.g., between BoNT/E and BoNT/F) up to about 66% at the amino acid level, (e.g., for BoNT/A vs BoNT/C or D). Thus, BoNT serotypes differ from each other by about 35-66% at the amino acid level.

The term “BoNT subtype” (e.g., a BoNT/A1 subtype) refers to botulinum neurotoxin gene sequences of a particular serotype (e.g., A, B, C, D, E, F, etc.) that differ from each other sufficiently to produce differential antibody binding. The subtypes may differ from each other by at least 2.5%, by at least 5%, by at least 10%, by at least 15% or up to about at least 20% at the amino acid level. The subtypes differ from each other by no more than 35%, by no more than 31.6%, by no more than 30%, or 25%, by less than about 20% or 16% at the amino acid level. BoNT subtypes may differ from each other by at least 2.6%, by at least 3%, and by at least 3.6% at the amino acid level. BoNT subtypes typically differ from each other by less than about 31.6%, by less than about 16%, at the amino acid level, other by less than about 31.6%, by less than about 16%, at the amino acid level.

An “anti-BoNT antibody” refers to an antibody that, specifically binds a BoNT polypeptide with a K_{D} less than 10⁻⁷, less than 10⁻⁸, less than 10⁻⁹, less than 10⁻¹⁰, less than 10⁻¹¹, or less than 10⁻¹² orless.

“Neutralization” refers to a measurable decrease in the toxicity and/or circulating level of a Botulinum neurotoxin (e.g., BoNT/A or BoNT/B).

“Potency” refers to the degree of protection from challenge with BoNT. This can be measured/quantified for example, as an increase in the LD₅₀ of a Botulinum neurotoxin (BoNT). In toxicology, the median lethal dose, LD₅₀ (abbreviation for “Lethal Dose, 50%”), or LCt₅₀ (Lethal Concentration & Time) of a toxic substance or radiation is the dose required to kill half the members of a tested population. The LD₅₀ is usually expressed as the mass of substance administered per unit mass of test subject, such as grams of substance per kilogram of body mass. Stating it this way allows the relative toxicity of different substances to be compared, and normalizes for the variation in the size of the animals exposed (although toxicity does not always scale simply with body mass). Typically, the LD₅₀ of a substance is given in milligrams per kilogram of body weight. In the case of some toxins, the LD₅₀ may be more conveniently expressed as micrograms per kilogram (μg/kg) of body mass.

The term “high affinity” when used with respect to an antibody refers to an antibody that specifically binds to its target(s) with an affinity (K_{D}) of at least about 10-6 M, at least about 10-7 M, at least about 10-8 M, preferably at least about 10-9 M, at least about 10-10 M, and at least about 10-11 M. “High affinity” antibodies may have a K_{D} that ranges from about 1 nM to about 5 pM.

The terms “polypeptide”, “peptide”, or “protein” are used interchangeably herein to designate a linear series of amino acid residues connected one to the other by peptide bonds between the alpha-amino and carboxy groups of adjacent residues. The amino acid residues are usually in the natural “L” isomeric form. However, residues in the “D” isomeric form can be substituted for any L-amino acid residue, as long as the desired functional property is retained by the polypeptide. In addition, the amino acids, in addition to the 20 “standard” amino acids, include modified and unusual amino acids, which include, but are not limited to those listed in 37 CFR (§1.822(b)(4)). Furthermore, it should be noted that a dash at the beginning or end of an amino acid residue sequence indicates either a peptide bond to a further sequence of one or more amino acid residues or a covalent bond to a carboxyl or hydroxyl end group. However, the absence of a dash should not be taken to mean that such peptide bonds or covalent bond to a carboxyl or hydroxyl end group is not present, as it is conventional in representation of amino acid sequences to omit such.

“Antibody” encompasses antigen-binding proteins having one or more polypeptides that can be genetically encodable by immunoglobulin genes, or fragments of immunoglobulin genes, and which bind an antigen of interest.

Antibodies of the present disclosure include “heavy chain-only” antibodies, which are also referred to as “heavy chain antibodies”, “HCAbs”, or “V_{H}H”, and antigen-binding fragments thereof. Antigen-binding fragments of HCAbs encompass, for example, Fab′, (Fab′)2, and “single-domain antibodies” (dAbs, also referred to as nanobodies). Heavy-chain only antibodies can be found naturally in camelids (e.g., llamas, camels) and can be produced through recombinant techniques, details of which are described later below. Naturally occurring HCAbs are antibodies are composed of two heavy chain polypeptides and thus lack light chain polypeptides found in naturally-occurring tetrameric antibodies. The heavy chains of HCAbs are composed of a variable region (V_{H}) and a constant region (C_{H}), where the V_{H} shares an organization structure of V_{H} of tetrameric antibodies, and is composed of framework regions and three complementarity determining regions (CDRs).

Alternatively, “antibody” can refer to single chain antibodies, which can encompass that contain two heavy chains linked together as a single polypeptide, or can encompass a heavy chain and a light chain linked together, as a single polypeptide (the latter of which may be referred to as a “scFv”).

“Antibody” can encompasse intact immunoglobulins as well antigen-binding fragments of antibodies. Thus, the term “antibody”, as used herein also includes an antigen-binding portion of an antibody, which can be produced by the modification of whole antibodies or synthesized de novo using recombinant DNA methodologies. Examples include, but are not limited to, Fab′, (Fab′)₂, scFv, and nanobodies. “Fab′” as used herein refers to a minimal antigen-binding portion of an antibody that lacks an Fc portion (e.g., a monomer of a V_{H} of a HCab or a heterodimer of a V_{H}/V_{L} pair of a tetrameric antibody).” (Fab′)₂″ refers to Fab molecules that are covalently linked, usually covalently linked as found in nature, which which lack an Fc portion.

An example of an antibody is one having a structural unit composed of one or two pairs of polypeptide chains. Where the antibody is a heavy chain-only antibody, the antibody contains heavy chain but not light chain.

Tetrameric antibodies refers to antibodies composed of two pairs of polypeptides, where each pair includes one “light” chain polypeptide and one “heavy” chain polypeptide. The terms variable light chain (V_{L}) and variable heavy chain (V_{H}) refer to the portions of the light and heavy chains that contain the CDRs, respectively. Light chains can be classified according to their constant regions, which can be kappa or lambda. Heavy chains can be classified according to their constant regions, which can be gamma, mu, alpha, delta, or epsilon, which in turn define the immunoglobulin classes, IgG, IgM, IgA, IgD and IgE, respectively.

It should be noted that while various antibody fragments may be defined in terms of the digestion of an intact antibody, one of skill will appreciate that such fragments may be synthesized de novo either chemically or by utilizing recombinant DNA methodology.

The term “antibody” encompasses polyclonal and monoclonal antibodies, and further encompasses antibodies of any class (e.g., IgM, IgG, and subclasses thereof). “Antibody” also encompasses hybrid antibodies, bispecific antibodies, heteroantibodies, chimeric antibodies, humanized antibodies, and functional fragments thereof which retain antigen binding. “Bispecific antibodies” may resemble single antibodies (or antibody fragments) but have two different antigen binding sites (variable regions). Heteroantibodies refers to two or more antibodies, or antibody binding fragments (e.g., Fab) linked together, each antibody or fragment having a different specificity. The antibodies may be conjugated to other moieties, and/or may be bound to a support (e.g., a solid support), such as a polystyrene plate or bead, test strip, and the like.

An immunoglobulin heavy or light chain variable region is composed of a “framework” region (FR) interrupted by three hypervariable regions, also called “complementarity determining regions” or “CDRs”. The extent of the framework region and CDRs have been defined (see, “Sequences of Proteins of Immunological Interest,” E. Kabat et al., U.S. Department of Health and Human Services, (1991 and Lefranc et al. IMGT, the international ImMunoGeneTics information system®. Nucl. Acids Res., 2005, 33:D593-D597)). A detailed discussion of the IMGTS system, including how the IMGTS system was formulated and how it compares to other systems, is provided on the World Wide Web at imgt.cines.fr/ textes/ IMGTScientificChart/ Numbering/IMGTnumberingsTable.html. The sequences of the framework regions of different light or heavy chains are relatively conserved within a species. The framework regions of an antibody serve to position and align the CDRs. The CDRs are primarily responsible for binding to an epitope of an antigen. All CDRs and framework provided by the present disclosure are defined according to Kabat et al, supra, unless otherwise indicated.

An “antigen-binding site” or “binding portion” refers to the part of an immunoglobulin molecule that participates in antigen binding. In a HCAb, the antigen binding site is provided by amino acid residues of the N-terminal variable (“V”) regions of the heavy chain (“V_{H}”). Where the antibody contains light chains, the variable reiongs of the light chains (“V_{L}”) with the V_{H} can also determine antigen binding. Three highly divergent stretches within the V regions are referred to as “hypervariable regions” which are interposed between more conserved flanking stretches known as “framework regions” or “FRs”. Thus, the term “FR” refers to amino acid sequences that are naturally found between and adjacent to hypervariable regions in immunoglobulins. Hypervariable regions mediate recognition and binding of the target antigen and are referred to as “complementarity determining regions” or “CDRs” and are characterized, for example by Kabat et al. Sequences of proteins of immunological interest, 4th ed. U.S. Dept. Health and Human Services, Public Health Services, Bethesda, Md. (1987).

An “Aa1 antibody” refers to an antibody expressed by clone Aa1 or to an antibody synthesized in other manners, but having the same CDRs and optionally, the same framework regions as the antibody expressed by clone Aa1. Similarly, antibodies Aa1, A26, A3, A16, A23, A10, Aa12, Aa6, Aa9, A8, A21, A19, Aa8, Aa5, Aa11, A8.1a, B01, B04, B12, B22, Bc1, Bc2, Bc3, Bc4, Bc5, Bc6, Bc7, Bc8, Bc9, Bc10, Bc11, Bc12, Bc13, or Bc14, and the like refer to antibodies expressed by the corresponding clone(s) and/or to antibodies synthesized in other manners, but having the same CDRs and optionally, the same framework regions as the referenced antibodies.

As used herein, the terms “immunological binding” and “immunological binding properties” refer to the non-covalent interactions of the type which occur between an immunoglobulin molecule and an antigen for which the immunoglobulin is specific. The strength or affinity of immunological binding interactions can be expressed in terms of the dissociation constant (K_{D}) of the interaction, wherein a smaller K_{D} represents a greater affinity. Immunological binding properties of selected polypeptides can be quantified using methods well known in the art. One such method entails measuring the rates of antigen binding site/antigen complex formation and dissociation, wherein those rates depend on the concentrations of the complex partners, the affinity of the interaction, and on geometric parameters that equally influence the rate in both directions. Thus, both the “on rate constant” (kₒₙ) and the “off rate constant” (k_{off}) can be determined by calculation of the concentrations and the actual rates of association and dissociation. The ratio of k_{off}/kₒₙ enables cancellation of all parameters not related to affinity and is thus equal to the equilibrium dissociation constant K_{D} (see, generally, Davies et al. Ann. Rev. Biochem.1990, 59: 439-15 473).

A “BoNT-inhibitory antibody” refers to an antibody that binds to one or more Botulinum neurotoxin(s) (e.g., BoNT/A1, BoNT/B1, BoNT/B2, BoNT/E1, etc.) and that by so-binding reduces the efficiency of BoNT neurotoxin to cleave its substrate (e.g. human SNAP25). Thus, a “BoNT/A-inhibitory antibody”, as used herein refers to an antibody that specifically binds to a BoNT/A polypeptide (e.g, a BoNT/A Lc) so as to reduce efficiency of BoNT/A in cleavage of its substrate. An example of such an antibody is one that binds to an Lc domain of a BoNT/A polypeptide and prevents BoNT/A Lc from cleaving SNAP25 as efficiently as BoNT/A Lc in the absence of the antibody. Reduced efficiency in substrate cleavage can be measured as an increase in the time for BoNT to convert its substrate to the resulting cleavage products or decrease in the total amount of cleavage products once equilibrium has been reached. Details will be described later in the examples section. Antibodies derived from BoNT-inhibitory antibodies include, but are not limited to, the antibodies whose sequence is expressly provided herein.

An “epitope” is a site on an antigen (e.g. BoNT) to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

“Isolated” refers to an entity of interest that is in an environment different from that in which the compound may naturally occur. An “isolated” compound is separated from all or some of the components that accompany it in nature and may be substantially enriched. “Isolated” also refers to the state of a compound separated from all or some of the components that accompany it during manufacture (e.g., chemical synthesis, recombinant expression, culture medium, and the like).

A single chain Fv (“scFv”) polypeptide is a covalently linked V_{H}::V_{L} heterodimer which may be expressed from a nucleic acid including V_{H}- and V_{L}-encoding sequences either joined directly or joined by a peptide-encoding linker (Huston, et al. (1988) Proc. Nat. Acad. Sci. USA, 85: 5879-5883). A number of structures are available for converting the light and heavy polypeptide chains from an antibody V region into an scFv molecule which will fold into a three dimensional structure substantially similar to the structure of an antigen-binding site. See, e.g. U.S. Pat. Nos. 5, 091,513 and 5,132,405 and 4,956,778.

Recombinant design methods may be used to develop suitable chemical structures (linkers) for converting two heavy and light polypeptide chains from an antibody variable region into a scFv molecule which will fold into a three-dimensional structure that is substantially similar to native antibody structure.

Design criteria include determination of the appropriate length to span the distance between the C-terminal of one chain and the N-terminal of the other, wherein the linker is generally formed from small hydrophilic amino acid residues that do not tend to coil or form secondary structures. Such methods have been described in the art. See, e.g., U.S. Pat. Nos. 5,091,513 and 5,132,405 to Huston et al.; and U.S. Pat. No. 4,946,778 to Ladner et al.

The phrase “specifically binds to” or “specifically immunoreactive with”, when referring to an antibody refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to a protein under such conditions may require an antibody that is selected for its specificity for a particular protein. For example, BoNT/B-inhibitory antibodies can be raised to BoNT/B protein(s) that specifically bind to BoNT/B protein(s), and not to other proteins present in a tissue sample. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988) Antibodies, A Laboratory Manual, Cold Spring Harbor Publications, New York, for a description of immunoassay formats and conditions that can be used to determine specific immunoreactivity.

The term “conservative substitution” is used in reference to proteins or peptides to reflect amino acid substitutions that do not substantially alter the activity (specificity or binding affinity) of the molecule. Typically conservative amino acid substitutions involve substituting one amino acid for another amino acid with similar chemical properties (e.g. charge or hydrophobicity). The following six groups each contain amino acids that are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

### DETAILED DESCRIPTION

This disclosure provides antibodies that specifically bind to botulinum neurotoxin (BoNT), and inhibit the catalytic activity of BoNT, and includes antibodies that inhibit BoNT catalytic activity by binding at an exosite of the toxin (e.g., rather than at a catalytic site of a BoNT). Anti-BoNT antibodies that bind BoNT/A Lc alpha-exosite are encompassed by the present disclosure. Thus, the present disclosure provides antibodies that bind to BoNT/A and inhibit the catalytic activity of BoNA Lc, leading to decreased efficiency of BoNT/A in cleavage of its substrate SNAP25. The subject antibodies may also bind to and inhibit the activity of BoNT of serotypes having the same natural substrate as that of BoNT/A (e.g. SNAP25).

An example of an antibody of the present disclosure is the Aa1 V_{H}H, which binds via alpha helices in the CDR1 and CDR3 to the BoNT/A Lc alpha-exosite groove in a manner similar to an alpha-helix in the BoNT/A belt and to the alpha-helix in the SNAP25 substrate. A number of the amino acid side chains in the V_{H}H which contact the BoNT/A Lc are the same contact side chains in SNAP25.

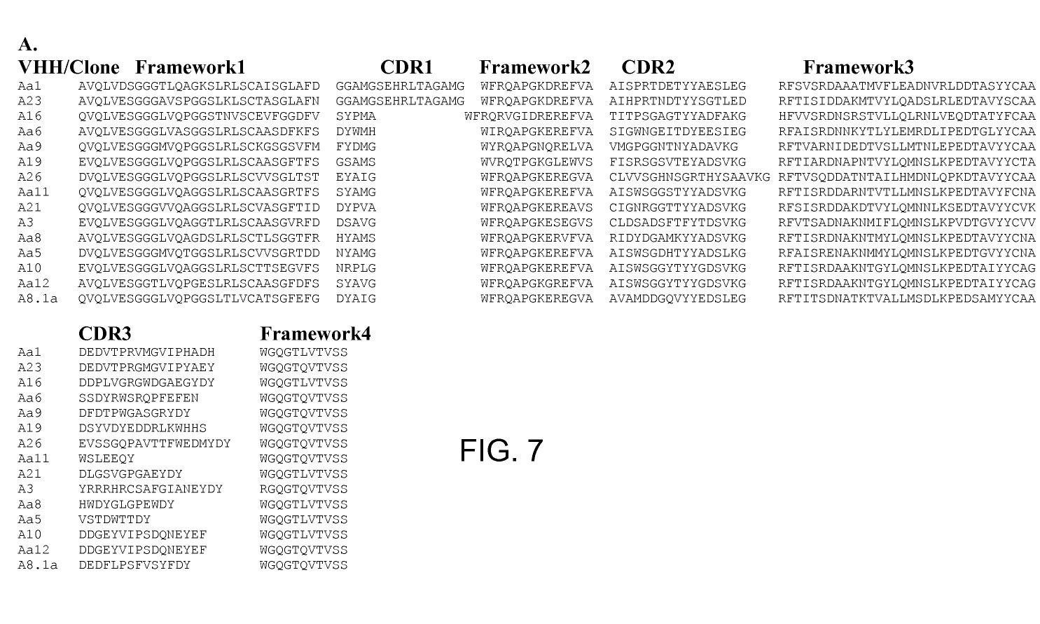

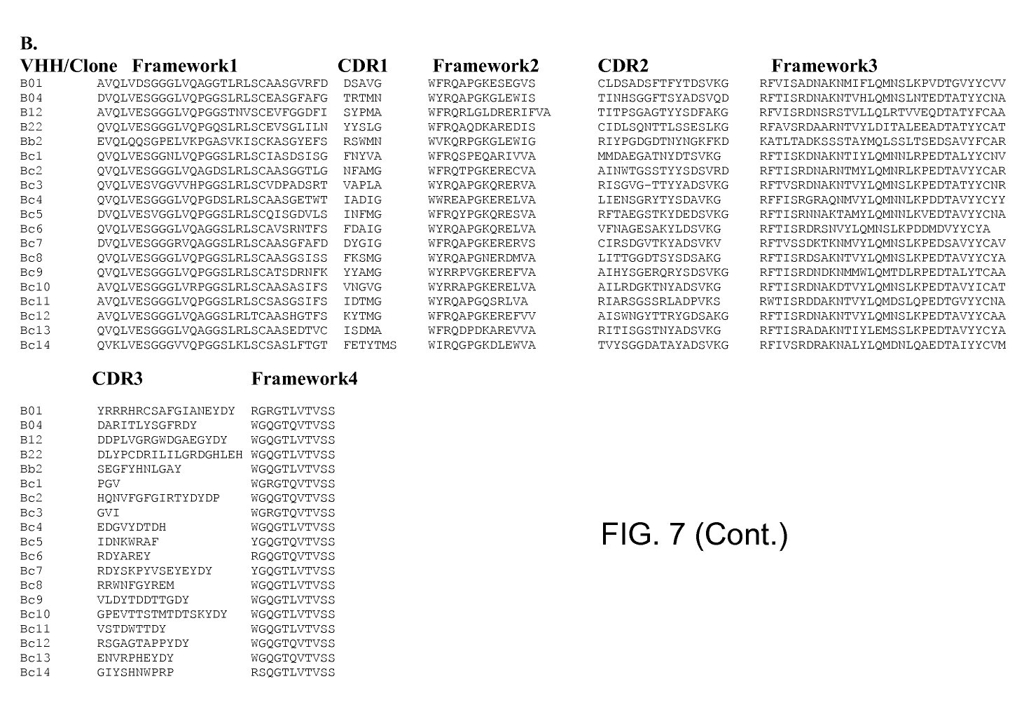

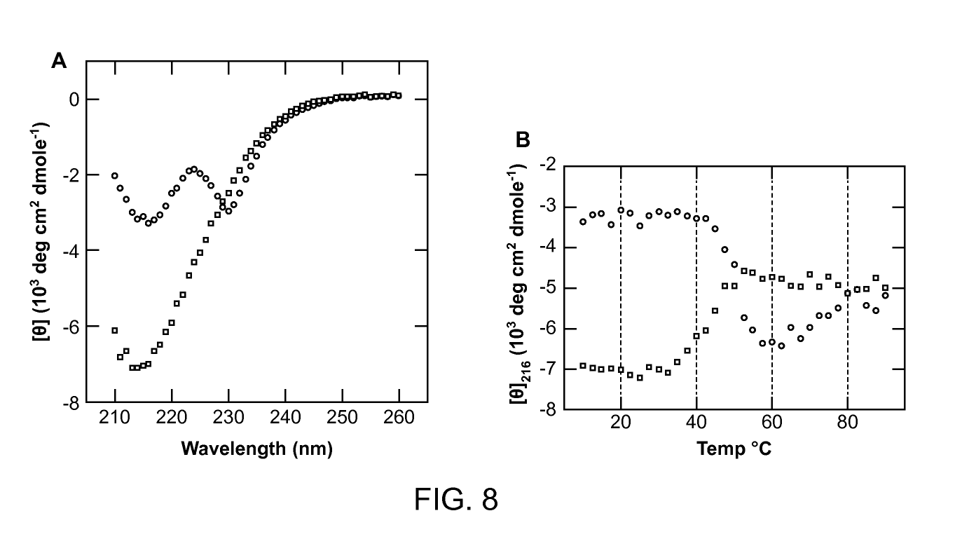

The present disclosure also provides compositions that include one or more different antibodies selected from the antibodies described herein (see, e.g., FIG. 7) and/or antibodies comprising one or more CDRs from these antibodies, and/or one or more antibodies comprising mutants or derivatives of these antibodies. The composition may include one or more antibodies, such as Aa1, A26, A3, A16, A23, A10, Aa12, Aa6, Aa9, A8, A21, A19, Aa8, Aa5, Aa11, A8.1a, B01, B04, B12, B22, Bc1, Bc2, Bc3, Bc4, Bc5, Bc6, Bc7, Bc8, Bc9, Bc10, Bc11, Bc12, Bc13, or Bc14.

Compositions contemplated herein may also include antibodies selected from those described in U.S. Pat. No. 7,563,874, US Pat Pub. No. 20080124328, PCT Pub No. WO/2009/008916, and PCT Application No. PCT/US09/52314.

As the antibodies of the present disclosure act to bind and inhibit botulinum neurotoxins, they are useful in the treatment of pathologies associated with botulinum neurotoxin poisoning and in the reversal of symptoms caused by infection of the toxin. The treatments can involve administering to the poisoned organism (e.g. human or non-human mammal) a quantity of one or more antibodies sufficient to inhibit (e.g. mitigate or eliminate) the symptoms of BoNT poisoning.

The treatment can be applicable in acute cases (e.g. where vital capacity is less than 30-40 percent of predicted and/or paralysis is progressing rapidly and/or hypoxemia with absolute or relative hypercarbia is present. These antibodies can also be used to treat early cases with symptoms milder than indicated (to prevent progression) or even prophylactically (a use the military envisions for soldiers going in harm's way). Treatment with the antibodies of the present disclosure can be provided as an adjunct to other therapies (e.g. antibiotic treatment).

The antibodies provided by this disclosure can also be used for the rapid detection/diagnosis of botulism.

### Botulinum Neurotoxin (BoNT)-Inhibitory Antibodies.

BoNT antibodies may be selected based on their affinity to one or more BoNT subtypes. A number of subtypes are known for each BoNT serotype. Thus, for example, BoNT/A subtypes include, but are not limited to, BoNT/A1, BoNT/A2, BoNT/A3, BoNT/A4, BoNT/A5, and the like. It is also noted, for example, that the BoNT/A1 subtype includes, but is not limited to 62A, NCTC 2916, ATCC 3502, and Hall hyper (Hall Allergan) and are identical (99.9-100% identity at the amino acid level.) and have been classified as subtype A1. The BoNT/A2 sequences (Kyoto-F and FRI-A2H) (Willems, et al. (1993) Res. Microbiol. 144:547-556) are 100% identical at the amino acid level. Another BoNT/A subtype, (that we are calling A3) is produced by a strain called Loch Maree that killed a number of people in an outbreak in Scotland. BoNT/A subtypes A1 to A4 bind SNAP25 with similar affinity but have different catalytic capacities for SNAP25 cleavage (Henkel et al, Biochemistry (2009) 48(11): 2522-28).

Antibodies of the present disclosure include those that bind BoNT/A and/or BoNT/B. Other BoNT serotypes that share similar epitopes and/or substrates as those of BoNT/A and BoNT/B can also be binding targets of subject antibodies. Examples of subject antibodies include those that can bind to BoNT/A Lc and inhibit the endopeptidase activity of BoNT/A Lc fragment. Antibodies that bind to BoNT/A and inhibit the catalytic activity thereof can also bind to other BoNT serotypes (BoNT/C and E) that have the same natural substrate as that of BoNT/A (e.g. SNAP25). Similarly, antibodies that bind to BoNT/B and inhibit the catalytic activity thereof can also bind to other BoNT serotypes (e.g. BoNT/D, F, and G) that have the same natural substrate as that of BoNT/A (e.g. VAMP).

When bound to BoNT, a subject antibody can decrease the amount of cleavage products derived from a substrate (e.g. SNAP25), compare to a cleavage reaction in the absence of the subject antibody. Accordingly, when contacted with the antibody of the present disclosure, BoNT does not cleave its substrates efficiently.

When complexed with BoNT/A Lc, certain antibodies of the present disclosure are found to contact a groove on a surface of the BoNT/A Lc fragment. The groove with which the antibodies make contact is also named the alpha-exosite (α-exosite), which is the site of binding of the natural substrate of BoNT, SNAP25. Certain antibodies of those exemplified herein have one or more CDR that contacts an α-exosite of BoNT/A Lc domain. An example of CDR that can make such contact is the V_{H} CDR1 of Aa1. Accordingly, antibodies having an amino acid sequence of V_{H} CDR1 of Aa1 can possess the property of inhibiting the catalytic activity of BoNT (e.g. BoNT/A Lc). Such antibodies may be provided as heavy chain-only antibodies (HCAb or dAb), which is also referred to herein as V_{H}H. Since the natural substrate of BoNT/C and BoNT/E also includes SNAP25 as that of BoNT/A, antibodies that can inhibit the catalytic activity of BoNT/A Lc and/or bind to the α-exosite of BoNT/A can also have similar inhibition and binding properties when complexed with BoNT/C or BoNT/E. Accordingly, the subject antibodies can also target BoNT/C or BoNT/E in the same fashion as BoNT/A, as described herein.

Additional examples of subject antibodies encompass those that have at least one CDR that is at least 87%, at least 93%, up to 100% amino acid sequence identity with the amino acid sequence of a V_{H} CDR of antibodies shown in FIG. 7 (e.g. V_{H} CDR1 of Aa1). The subject antibody can also include more than one CDR from any V_{H} CDRs of antibodies shown in FIG. 7, and combinations therein, such that each CDR in the subject antibody may be independently selected from an antibody shown in FIG. 7. For example, an antibody may contain a V_{H} CDR1 of Aa1, a V_{H} CDR1 and a V_{H} CDR3 from Aa12; all three V_{H} CDRs of Aa1; or a V_{H} CDR1 from Aa1 and a V_{H} CDR3 from A23, etc. Antibodies of the present disclosure of particular interest are HCAbs composed of CDRs of a V_{H} disclosed herein.

Optionally, antibodies can be provided by associating a a V_{H}H (dAb) with a light chain, e.g., an irrelevant light chain or a light chain that increases target antigen affinity relative to a V_{H}H that are not linked to a light chain. The light chain can also impart specificity that the V_{H}H alone would not have alone to result in a bi-specific antibody. The light chain can be linked noncovalently with a VH having any VH CDRs shown in FIG. 7 or covalently as a single-chain antibody (scFV).

The present disclosure also provides homodimeric and heterodimeric antibodies composed of the same or different V_{H} of a V_{H}H disclosed herein.

Properties of examples of antibodies of the present disclosure that bind to BoNT/A or a BoNT serotype having the same substrate as BoNT/A are listed in Table 1 below.

| V_{H}H | | |
|---|---|---|
| clone | | Yeast-displayed V_{H}H K_{D} |
| name | Sequences of CDR3 | for BoNT/A Lc (nM) |
| A26 | EVSSGQPAVTTFWEDMYDY | 8.7 |
| A3 | YRRRHRCSAFGIANEYDY | 6.63 |
| A16 | DDPLVGRGWDGAEGYDY | 4.04 |
| Aa1^{a,b} | DEDVTPRVMGVIPHADH | 0.03 |
| A23^{a,b} | DEDVTPRGMGVIPYAEY | 16.94 |
| A10^{c} | DDGEYVIPSDQNEYEF | 76.34 |
| Aa12^{c} | DDGEYVIPSDQNEYEF | 30.27 |
| Aa6^{a} | SSDYRWSRQPFEFEN | 0.52 |
| Aa9^{a} | DFDTPWGASGRYDY | 4.08 |
| A8a | DEDLLPSFVSDFDY | 229.9 |
| A21 | DLGSVGPGAEYDY | 60.74 |
| A19^{a} | DSYVDYEDDRLK | 4.65 |
| Aa8^{a} | HWDYGLGPE | 112.09 |
| Aa5^{a} | VSTDWTTDY | 207 |
| Aa11 | WSLEEQY | 76.08 |

^{a}V_{H}H which inhibit the catalytic activity of BoNT/A Lc.
^{b}V_{H}H Aa1 and A23 have the same CDR1 sequence and highly related CDR3 sequence.
^{c}V_{H}H A10 and Aa12 have the same CDR3 sequence but different CDR1 and CDR2 sequence

The amino acid sequences of the variable heavy (V_{H}) for a number of antibodies that bind BoNT/A and BoNT/B are illustrated in FIG. 7.

It will be appreciated that the amino acid sequence of a CDR can also be defined using alternative systems, which will be readily apparent to and applied by the ordinarily skilled artisan (see, “Sequences of Proteins of Immunological Interest,” E. Kabat et al., U.S. Department of Health and Human Services, (1991 and Lefranc et al. IMGT, the international ImMunoGeneTics information system. Nucl. Acids Res., 2005, 33, D593-D597)). A detailed discussion of the IMGTS system, including how the IMGTS system was formulated and how it compares to other systems, is provided on the World Wide Web at imgt.cines.fr/ textes/ IMGTScientificChart/ Numbering/IMGTnumberingsTable.html. All amino acid sequences of CDR in the present disclosure are defined according to Kabat et al., supra, unless otherwise indicated.

The variable heavy chains disclosed herein can be joined directly or through a linker (e.g., (Gly₄Ser)₃, SEQ ID NO:1) to form a single-chain antibody. The various CDRs and/or framework regions can be used to form human antibodies, chimeric antibodies, antibody fragments, and the like.

Anti-BoNT antibodies of the present disclosure have a binding affinity (K_{D}) for a BoNT protein of at least 10⁻⁶, at least 10⁻⁷, 10⁻⁸, at least 10⁻⁹, at least 10⁻¹⁰, at least 10⁻¹¹, up to 10⁻¹²M or less. Some examples of K_{D}s (M⁻¹) for BoNT/A Lc are shown in Table 1 above and fall in the following ranges: between 5×10⁻¹¹ to 3×10⁻¹⁰, between 4×10⁻¹⁰ to 2×10⁻¹⁰, between 7×10⁻¹⁰ to 1×10⁻⁹, between 8×10⁻¹⁰ to 5×10⁻⁹, between 1×10⁻⁹ to 3×10⁻⁹, between 4×10⁻⁹ to 2×10⁻⁸, 1×10⁻⁸ to 8×10⁻⁸, and 5×10⁻⁷ to 1×10⁻⁷.

The antibodies encompass those that bind to an epitope of BoNT bound by an antibody containing one or more of the CDRs set forth FIG. 7. Epitopes bound by an antibody may be described by a specific BoNT domain and/or the residues therein that contribute to the interaction between the antibody and a BoNT protein. Antibodies that bind an alpha-exosite of a BoNT Lc, e.g., an alpha-exosite of BoNT/A Lc, are encompassed by the present disclosure.

The ability of a particular antibody to recognize the same epitope as another antibody can be determined by the ability of one antibody to competitively inhibit binding of the second antibody to the antigen. Competitive inhibition of binding may also be referred to as cross-reactivity of antibodies. Any of a number of competitive binding assays can be used to measure competition between two antibodies to the same antigen. For example, a sandwich ELISA assay can be used for this purpose. Additional methods for assaying for cross-reactivity are described later below.

An antibody is considered to competitively inhibit binding of a second antibody, if binding of the second antibody to the antigen is reduced by at least 30%, usually at least about 40%, 50%, 60% or 75%, and often by at least about 90%, in the presence of the first antibody using any of the assays used to assess competitive binding.

Antibodies of the present disclosure include those that compete for binding to a Botulinum neurotoxin at the α-exosite of BoNT/A Lc domain with one or more antibodies disclosed herein as BoNT/A binders (see, e.g., FIG. 7, panel A) and/or compete with SNAP25 for binding to BoNT/A. Similarly, the subject antibodies also include those that compete for binding to any BoNT serotypes that have the same natural substrate (e.g. SNAP25) as BoNT/A with BoNT/A binders shown in panel A of FIG. 7. The antibodies of the present disclosure also include those that compete for binding to BoNT/B with one or more antibodies disclosed herein as BoNT/B binders (see, e.g., FIG. 7, panel B).

Accordingly, antibodies provided by the present disclosure encompass those that compete for binding to a BoNT/A, BoNT/B, BoNT/C, or BoNT/E with an antibody that includes one or more of the V_{H} CDRs set forth in FIG. 7. Antibodies provided by the present disclosure also encompass those that compete for binding to a BoNT with an antibody that includes one or more of the V_{H} CDRs set forth in FIG. 7. Additional antibodies may encompass those that compete for binding to a BoNT/A (or BoNT/C or BoNT/E) with an antibody with one or more CDRs set forth in FIG. 7.

For example, an antibody may have the binding specificity of an antibody having one or more V_{H} CDRs or full length V_{H} as set forth in FIG. 7. An antibody of the present disclosure may therefore contain a CDR as set forth in a V_{H} sequence shown in FIG. 7 and, additionally, may have at least 80% identity, at least 85%, at least 90%, or at least 95% amino acid sequence identity to a full-length V_{H} sequence. For example, an antibody may contain the CDRs of a V_{H} sequence and human framework sequences set forth in FIG. 7.

### Preparation of BoNT Inhibitory Antibodies.

### Recombinant Expression of BoNT-Inhibitory Antibodies.

Using the information provided herein, the botulinum neurotoxin-inhibitory antibodies of the present disclosure are prepared using standard techniques well known to those of skill in the art.

For example, the polypeptide sequences provided herein (see, e.g., Table 1 and/or FIG. 7) can be used to determine appropriate nucleic acid sequences encoding the BoNT-inhibitory antibodies and the nucleic acids sequences then used to express one or more BoNT-inhibitory antibodies. The nucleic acid sequence(s) can be optimized to reflect particular codon “preferences” for various expression systems according to standard methods well known to those of skill in the art.

Using the sequence information provided, the nucleic acids may be synthesized according to a number of standard methods known to those of skill in the art. Oligonucleotide synthesis, is preferably carried out on commercially available solid phase oligonucleotide synthesis machines (Needham-VanDevanter et al. (1984) Nucleic Acids Res. 12:6159-6168) or manually synthesized using, for example, the solid phase phosphoramidite triester method described by Beaucage et. al. (1981) Tetrahedron Letts. 22(20): 1859-1862.

Once a nucleic acid encoding an anti-BoNT antibody is synthesized it can be amplified and/or cloned according to standard methods. Molecular cloning techniques to achieve these ends are known in the art. A wide variety of cloning and in vitro amplification methods suitable for the construction of recombinant nucleic acids are known to persons of skill in the art.

Once the nucleic acid for an anti-BoNT antibody is isolated and cloned, one can express the nucleic acid in a variety of recombinantly engineered cells known to those of skill in the art. Examples of such cells include bacteria, yeast, filamentous fungi, insect (especially employing baculoviral vectors), and mammalian cells. Pichia and mammalian cell lines (e.g., immortalized human cell lines) are contemplated.

Expression of natural or synthetic nucleic acids encoding anti-BoNT antibodies can be be achieved by operably linking a nucleic acid encoding the antibody to a promoter (which is either constitutive or inducible), and incorporating the construct into an expression vector. The vectors can be suitable for replication and integration in prokaryotes, eukaryotes, or both. Typical cloning vectors contain transcription and translation terminators, initiation sequences, and promoters useful for regulation of the expression of the nucleic acid encoding the anti-BoNT antibody. The vectors optionally comprise generic expression cassettes containing at least one independent terminator sequence, sequences permitting replication of the cassette in both eukaryotes and prokaryotes, i.e., shuttle vectors, and selection markers for both prokaryotic and eukaryotic systems.

To obtain high levels of expression of a cloned nucleic acid it is common to construct expression plasmids which typically contain a strong promoter to direct transcription, a ribosome binding site for translational initiation, and a transcription/translation terminator. Examples of regulatory regions suitable for this purpose in E. coli are the promoter and operator region of the E. coli tryptophan biosynthetic pathway, the leftward promoter of phage lambda (P_{L}), and the L-arabinose (araBAD) operon. The inclusion of selection markers in DNA vectors transformed in E. coli is also useful. Examples of such markers include genes specifying resistance to ampicillin, tetracycline, or chloramphenicol. Expression systems for expressing anti-BoNT antibodies are available using, for example, E. coli, Bacillus sp. and Salmonella. E. coli systems may also be used.

The anti-BoNT antibodies produced by prokaryotic cells may require exposure to chaotropic agents for proper folding. During purification from, e.g., E. coli, the expressed protein is optionally denatured and then renatured. This can be accomplished, e.g., by solubilizing the bacterially produced antibodies in a chaotropic agent such as guanidine HCl. The antibody is then renatured, either by slow dialysis or by gel filtration. Alternatively, nucleic acid encoding the anti-BoNT antibodies may be operably linked to a secretion signal sequence such as pelB so that the anti-BoNT antibodies are secreted into the medium in correctly-folded form.

Methods of transfecting and expressing genes in mammalian cells are known in the art. Transducing cells with nucleic acids can involve, for example, incubating viral vectors containing anti-BoNT nucleic acids with cells within the host range of the vector. The culture of cells used in the present disclosure, including cell lines and cultured cells from tissue or blood samples is well known in the art.

The BoNT-inhibitory antibody gene(s) (e.g. BoNT-inhibitory V_{H}H gene) may be subcloned into the expression vector pUC119mycHis or pSYN3, resulting in the addition of a hexahistidine tag at the C-terminal end of the scFv to facilitate purification. Detailed protocols for the cloning and purification of certain BoNT-inhibitory antibodies are found, for example, in Amersdorfer et al. (1997) Infect. Immunity, 65(9): 3743-3752, and the like.

### Preparation of Whole Polyclonal or Monoclonal Antibodies.

Anti-BoNT antibodies may be selected to bind one or more epitopes bound by the antibodies described herein (e.g., Aa1, A26, A3, A16, A23, A10, Aa12, Aa6, Aa9, A8, A21, A19, Aa8, Aa5, Aa11, A8.1a, B01, B04, B12, B22, Bc1, Bc2, Bc3, Bc4, Bc5, Bc6, Bc7, Bc8, Bc9, Bc10, Bc11, Bc12, Bc13, or Bc14). Methods of making antibodies that specifically bind to a particular epitope are known in the art.

Polyclonal antibodies can be made using methods well known to those of skill in the art. In brief, an immunogen (e.g., BoNT/A, BoNT/B, BoNT/E, etc., e.g., a BoNT Lc, e.g., a BoNT/A Lc,) having an epitope specifically bound by antibodies expressed by clones Aa1, A26, A3, A16, A23, A10, Aa12, Aa6, Aa9, A8, A21, A19, Aa8, Aa5, Aa11, A8.1a, B01, B04, B12, B22, Bc1, Bc2, Bc3, Bc4, Bc5, Bc6, Bc7, Bc8, Bc9, Bc10, Bc11, Bc12, Bc13, or Bc14 disclosed herein is administered to a non-human animal, and antibodies obtained from the serum of the immunized animal. The animal's immune response to the immunogen preparation is monitored by taking test bleeds and determining the titer of reactivity to the polypeptide of interest. When appropriately high titers of antibody to the immunogen are obtained, blood is collected from the animal and antisera are prepared. Further fractionation of the antisera to enrich for antibodies reactive to the BoNT polypeptide is performed where desired. Antibodies that specifically bind to the inhibitory epitopes described herein can be selected from polyclonal sera using the selection techniques described herein.

Methods of producing monoclonal antibodies from various mammalian hosts, such as mice, rodents, primates, humans, etc. are known in the art. Summarized briefly, monoclonal antibody production using hybridomas may proceed by injecting an animal with an (e.g., BoNT/A, BoNT/B, BoNT/C, BoNT/E etc.) subsequences including, but not limited to subsequences comprising epitopes specifically bound by antibodies expressed by clones Aa1, A26, A3, A16, A23, A10, Aa12, Aa6, Aa9, A8, A21, A19, Aa8, Aa5, Aa11, A8.1a, B01, B04, B12, B22, Bc1, Bc2, Bc3, Bc4, Bc5, Bc6, Bc7, Bc8, Bc9, Bc10, Bc11, Bc12, Bc13, or Bc14 disclosed herein. The animal is then sacrificed and cells taken from its spleen, which are fused with myeloma cells. The result is a hybrid cell or “hybridoma” that is capable of reproducing antibodies in vitro. The population of hybridomas is then screened to isolate individual clones, each of which secretes a single antibody species to the immunogen. In this manner, the individual antibody species obtained are the products of immortalized and cloned single B cells from the immune animal generated in response to a specific site recognized on the immunogenic substance.

Alternative methods of immortalization include transformation with Epstein Barr Virus, oncogenes, or retroviruses, or other methods known in the art. Colonies arising from single immortalized cells are screened for production of antibodies of the desired specificity and affinity for the BoNT antigen, and yield of the monoclonal antibodies produced by such cells is enhanced by various techniques, including injection into the peritoneal cavity of a vertebrate (preferably mammalian) host. The antibodies of the present disclosure are used with or without modification, and include chimeric antibodies such as humanized murine antibodies.

Techniques for creating recombinant DNA versions of the antigen-binding regions of antibody molecules which bypass the generation of hybridomas are contemplated for the present BoNT (e.g., BoNT/A, BoNT/C, or BoNT/E) binding antibodies and fragments. DNA is cloned into a bacterial expression system. One example of a suitable technique uses a bacteriophage lambda vector system having a leader sequence that causes the expressed Fab protein to migrate to the periplasmic space (between the bacterial cell membrane and the cell wall) or to be secreted. One can rapidly generate and screen great numbers of functional Fab fragments for those which bind BoNT. Such BoNT binding agents (Fab fragments with specificity for a BoNT polypeptide) are specifically encompassed within the BoNT binding antibodies and fragments of the present disclosure. Other methods for screening and production of antibodies may employ one or more of display systems such as phage display, yeast display, ribosome, etc., and an antibody production system such as that derived from transgenic mice.

### Modification of BoNT Inhibitory Antibodies.

The present disclosure encompasses BoNT antibodies that are modified to provide a desired feature, e.g., to facilitate delivery to neurons in a subject, to increase serum half-life, etc.).

Modifications to facilitate the delivery of the subject antibodies or nucleic acid encoding thereof across cell membranes of cells (e.g. “transcytosis” of neurons) are known. Options include the non-neuron specific and neuron-specific delivery. The subject antibody may be provided as a fusion peptide along with a second peptide which promotes uptake of the peptide by neurons (e.g. neurons outside the central nervous system). For example, antibodies of the present disclosure can be provided as part of a fusion polypeptide with all or a fragment of the N-terminal domain of the HIV protein Tat, e.g., residues 1 72 of Tat or a smaller fragment thereof which can promote transcytosis. In other embodiments, the E2 peptide can be provided a fusion polypeptide with all or a portion of the antenopedia III protein. Any other peptides that are known to have transcytosis properties may also be used as a second peptide fused to the subject antibody (e.g. U.S. Pat. No. 6,248,558). Gene delivery methods are also contemplated herein to deliver nucleic acids that express the subject antibodies in cells.

Where delivery into the brain is desired, modification may be dependent on the strategy employed to deliver the subject antibodies. Some strategies may include (i) chemical delivery systems, such as lipid-mediated transport, the prodrug approach and the lock-in system; (ii) biological delivery systems, in which pharmaceuticals are re-engineered to cross the blood-brain barrier via specific endogenous transporters localized within the brain capillary endothelium; (iii) disruption of the blood-brain barrier, for example by modification of tight junctions, which causes a controlled and transient increase in the permeability of brain capillaries; (iv) the use of molecular Trojan horses, such as peptidomimetic monoclonal antibodies to transport large molecules (e.g. antibodies) across the blood-brain barrier; and (v) particulate drug carrier systems. See Patel et al (2009) CNS Drugs 23:35-58 for review. Neuron-specific targeting strategies based on receptor-ligand interactions include neuropeptides (e.g. neurotensin), neurotrophiins (e.g. nerve growth factor), and neurotoxins (e.g. tetanus toxin). Methods for delivering antibodies to the CNS are described, for example, in US 20090016959.

Adsorptive-mediated transcytosis (AMT) provides a means for brain delivery of antibodies across the blood-brain barrier. AMT-based drug delivery to the brain has been performed using cationic proteins and cell-penetrating peptides. Cationization is a chemical treatment that causes the conversion of superficial carboxyl groups on a protein into extended primary amino groups. This can be used to increase interactions of the antibody with the negative charges at the luminal plasma membrane of the brain endothelial cells. The cationized antibody can then undergo adsorptive mediated transcytosis through the blood-brain barrier. Antibodies can be cationized using various, synthetic (hexamethylenediamine) or naturally occurring (e.g., putrescine) polyamines (Herve et al (2008) AAPS J. 10: 455-72).

The BoNT-inhibitory antibody gene(s) (e.g. BoNT-inhibitory scFv gene) may be delivered into neurons using a variety of methods. Nonviral delivery methods are reviewed for example in Bergen et al (2008) Pharm Res. 25(: 983-98 and include: cationic polymers (e.g. polyethylimine); cationic lipids (e.g. 1,2-dioleoyl-3-trimethylammonium propane (DOTAP), N-methyl-4-(dioleyl)methylpyridinium (SAINT-2), 3β-[N-(N′,N′-dimethylaminoethane)-carbamoyl]cholesterol (DC-Chol), GS1, dioleoylphosphatidylethanolamine (DOPE), cholesterol or combinations thereof); PEGylated immunoliposomes (PILs), which consist of plasmid DNA encapsulated by PEG-modified neutral lipids; engineered polypeptides (e.g. recombinant fusion proteins based on the tetanus toxin fragment C, nerve growth factor -derived targeting peptides); nanoparticles; and naked DNA delivery. Viral gene delivery vehicles are reviewed in Davidoson & Breakefield (2003) Nature Rev. Neurosci. 4:353-364 and include adeno-associated virus and herpes simplex virus. Linkers can be used to join an antigen-binding portion of an antibody with a molecule of interest. Examples of linkers include polypeptide chains of alternating sets of glycine and serine residues, and may include glutamic acid and lysine residues inserted to enhance solubility. Such linkers are often referred to as “flexible linkers”. Examples include (Gly₄Ser)ₙ and ((Ser)₄Gly)ₙ (SEQ ID NO:35) where n is an integer of 1, 2, 3, 4 or more. Nucleotide sequences encoding such linker moieties can be readily provided using various oligonucleotide synthesis techniques known in the art (see, e.g., Sambrook, supra.).

### Selection of Inhibitory Antibodies.

Selection of anti-BoNT antibodies (whether produced by phage display, yeast display, immunization methods, hybridoma technology, etc.) involves screening the resulting antibodies for specific binding to an appropriate antigen(s). In the instant case, suitable antigens can include, but are not limited to BoNT/E1, BoNT/E2, BoNT/E3, BoNT/B1, BoNT/B2, BoNT/B3, BoNT/B4, BoNT/A1, BoNT/A2, and BoNT/A3. Use of Lc alpha-exosite as a target antigen is of particular interest.

The inhibitory antibodies may be selected for specific binding of an epitope recognized by one or more of the antibodies described herein, and can be further tested for activity in inhibiting cleavage of a BoNT substrate (e.g., as illustrated in the Examples below). Selection can be by any of a number of methods well known to those of skill in the art. In one example, selection is by immunochromatography (e.g., using immunotubes, Maxisorp, Nunc) against the desired target, e.g., BoNT/A, BoNT/B, etc. In a related example, selection is against a BoNT protein in a surface plasmon resonance system (e.g., BIAcore, Pharmacia) either alone or in combination with an antibody that binds to an epitope specifically bound by one or more of the antibodies described herein. Selection can also be done using flow cytometry for yeast display libraries.

### Humanized, Human Engineered or Human Antibody Production.

Anti-BoNT (e.g., BoNT/A) binding antibodies and fragments can be humanized or human engineered antibodies. As used herein, a humanized antibody, or antigen binding fragment thereof, is a recombinant polypeptide that comprises a portion of an antigen binding site from a non-human antibody and a portion of the framework and/or constant regions of a human antibody. A human engineered antibody or antibody fragment may be derived from a human or non-human (e.g., mouse) source that has been engineered by modifying (e.g., deleting, inserting, or substituting) amino acids at specific positions so as to alter certain biophysical properties or to reduce any detectable immunogenicity of the modified antibody in a human.

Humanized antibodies also encompass chimeric antibodies and CDR-grafted antibodies in which various regions may be derived from different species. Chimeric antibodies may be antibodies that include a non-human antibody variable region linked to a human constant region. Thus, in chimeric antibodies, the variable region is mostly non-human, and the constant region is human. CDR-grafted antibodies are antibodies that include the CDRs from a non-human “donor” antibody linked to the framework region from a human “recipient” antibody. For example, a CDR-grafted humanized antibody may comprise a heavy chain that comprises a contiguous amino acid sequence (e.g., about 5 or more, 10 or more, or even 15 or more contiguous amino acid residues) from the framework region of a human antibody (e.g., FR-1, FR-2, or FR-3 of a human antibody) or, optionally, most or all of the entire framework region of a human antibody.

Human engineered antibodies include for example “veneered” antibodies and antibodies prepared using HUMAN ENGINEERING™ technology (U.S. Pat. No. 5,869,619). HUMAN ENGINEERING™ technology is commercially available, and involves altering an non-human antibody or antibody fragment, such as a non-human (e.g., mouse, llama) or chimeric antibody or antibody fragment, by making specific changes to the amino acid sequence of the antibody so as to produce a modified antibody with reduced immunogenicity in a human that nonetheless retains the desirable binding properties of the original non-human antibodies. “Veneered” antibodies are non-human or humanized (e.g., chimeric or CDR-grafted antibodies) antibodies that have been engineered to replace certain solvent-exposed amino acid residues to reduce immunogenicity and/or enhance function. Veneering can be accomplished by any suitable engineering technique, including the use of the above-described HUMAN ENGINEERING™ technology.

### Nanobodies.

Nanobodies, also referred to as V_{H}H fragment or dAb, have a structure based n single chain antibodies such as those derived from camelids (e.g., llamas, camels), which are a homodimeric complex composed of a two heavy chains dimerized via their constant regions. The variable domains of these camelidae heavy chain antibodies are referred to as nanobodies. Isolated V_{H}H retain the ability to bind antigen with high specificity (see, e.g., Hamers-Casterman et al. (1993) Nature 363: 446-448). V_{H}H domains, or nucleotide sequences encoding them, can be derived from antibodies raised in Camelidae species, for example in camel, dromedary, llama, alpaca and guanaco. Other species besides Camelidae (e.g, shark, pufferfish) can produce functional antigen-binding heavy chain antibodies, from which (nucleotide sequences encoding) such naturally occurring V_{H}H can be obtained, e.g. using the methods described in US 2006/0211088.

Libraries of single VH domains have also been derived for example from VH genes amplified from genomic DNA or from mRNA from the spleens of immunized mice and expressed in E. coli (Ward et al. (1989) Nature 341: 544-546) and similar approaches can be performed using the V_{H} domains and/or the V_{L} domains described herein. The isolated single V_{H} domains are called “dAbs” or domain antibodies. A “dAb” is an antibody single variable domain (V_{H} or V_{L}) polypeptide that specifically binds antigen.

### UniBodies.

UniBodies are monovalent antibodies composed of one heavy and one light chain polypeptide, but lack the core hinge region found in naturally occurring tetrameric antibodies. Methods of producing UniBodies are described in W02007/059782 and Kolfschoten et al. (2007) Science 317: 1554-1557).

### Assaying for Cross-Reactivity at an Inhibitory Epitope.

The antibodies of the present disclosure encompass those that specifically bind to one or more epitopes (e.g. α-exosite) recognized by antibodies described herein (e.g., Aa1, A26, A3, A16, A23, A10, Aa12, Aa6, Aa9, A8, A21, A19, Aa8, Aa5, Aa11, A8.1a, B01, B04, B12, B22, Bc1, Bc2, Bc3, Bc4, Bc5, Bc6, Bc7, Bc8, Bc9, Bc10, Bc11, Bc12, Bc13, or Bc14, etc.). In other words, antibodies are cross-reactive with one of more of these antibodies. Means of assaying for cross-reactivity are well known to those of skill in the art (see, e.g., Dowbenko et al. (1988) J. Virol. 62: 4703-4711).

This can be ascertained by providing one or more isolated target BoNT polypeptide(s) (e.g. BoNT/A1, BoNT/A2, and/or BoNT/B, or recombinant domains of said toxin, such as L_{c}) attached to a solid support and assaying the ability of a test antibody to compete with, an antibody described herein for binding to the target BoNT peptide. Thus, immunoassays in a competitive binding format are preferably used for cross-reactivity determinations. For example, a BoNT/A and/or BoNT/B polypeptide may be immobilized to a solid support. Antibodies to be tested (e.g. generated by selection from a phage-display library) added to the assay compete with Aa1, A26, A3, A16, A23, A10, Aa12, Aa6, Aa9, A8, A21, A19, Aa8, Aa5, Aa11, A8.1a, B01, B04, B12, B22, Bc1, Bc2, Bc3, Bc4, Bc5, Bc6, Bc7, Bc8, Bc9, Bc10, Bc11, Bc12, Bc13, or Bc14, etc antibodies binding to the immobilized BoNT polypeptide(s). The ability of test antibodies to compete with the binding of the Aa1, A26, A3, A16, A23, A10, Aa12, Aa6, Aa9, A8, A21, A19, Aa8, Aa5, Aa11, A8.1a, B01, B04, B12, B22, Bc1, Bc2, Bc3, Bc4, Bc5, Bc6, Bc7, Bc8, Bc9, Bc10, Bc11, Bc12, Bc13, or Bc14, etc antibodies to the immobilized protein(s) are compared. The percent cross-reactivity above proteins is then calculated, using standard calculations.

If the test antibody competes with one or more of the Aa1, A26, A3, A16, A23, A10, Aa12, Aa6, Aa9, A8, A21, A19, Aa8, Aa5, Aa11, A8.1a, B01, B04, B12, B22, Bc1, Bc2, Bc3, Bc4, Bc5, Bc6, Bc7, Bc8, Bc9, Bc10, Bc11, Bc12, Bc13, or Bc14, etc antibodies and has a binding affinity comparable to or greater than about 1×10⁻⁶M, greater than about 1×10⁻⁷ M, or greater than about 1×10⁻⁸ M with the same target then the test antibody is expected to be a BoNT-inhibitory antibody.

Cross-reactivity may performed by using surface plasmon resonance in a BIAcore. In a BIAcore flow cell, the BoNT polypeptide(s) (e.g., BoNT/A and/or BoNT/B) are coupled to a sensor chip (e.g. CM5) as described in copending application No. 60/942,173, disclosure of which is incorporated herein by reference. With a flow rate of 5 μl/min, a titration of 100 nM to 1 μM antibody is injected over the flow cell surface for about 5 minutes to determine an antibody concentration that results in near saturation of the surface. Epitope mapping or cross-reactivity is then evaluated using pairs of antibodies at concentrations resulting in near saturation and at least 100 RU of antibody bound. The amount of antibody bound is determined for each member of a pair, and then the two antibodies are mixed together to give a final concentration equal to the concentration used for measurements of the individual antibodies. Antibodies recognizing different epitopes show an essentially additive increase in the RU bound when injected together, while antibodies recognizing identical epitopes show only a minimal increase in RU. Antibodies may be said to be cross-reactive if, when “injected” together they show an essentially additive increase (preferably an increase by at least a factor of about 1.4, more preferably an increase by at least a factor of about 1.6, and most preferably an increase by at least a factor of about 1.8 or 2.

Cross-reactivity may also be determined by incubating a yeast displayed dAbs with a BoNT domain polypeptide followed by incubation with an epitope-tagged dAb. Bound V_{H}H is detected with an antibody recognizing the epitope tag and the level of BoNT domain display quantitated by incubation with anti-SVS.

Cross-reactivity at the desired epitopes can ascertained by a number of other standard techniques (see, e.g., Geysen et al (1987) J. Immunol. Meth 102:259-274). This technique involves the synthesis of large numbers of overlapping BoNT peptides. The synthesized peptides are then screened against one or more of the prototypical antibodies (e.g., Aa1, etc.) and the characteristic epitopes specifically bound by these antibodies can be identified by binding specificity and affinity. The epitopes thus identified can be conveniently used for competitive assays as described herein to identify cross-reacting antibodies.

The peptides for epitope mapping can be conveniently prepared using “Multipin” peptide synthesis techniques (see, e.g., Geysen et al (1987) Science 235:1184-1190). Using the known sequence of one or more BoNT subtypes (see, e.g., Atassi et al. (1996) J. Prot. Chem. 7: 691-700 and references cited therein), overlapping BoNT polypeptide sequences can be synthesized individually in a sequential manner on plastic pins in an array of one or more 96-well microtest plate(s).

### Assaying for Inhibitory Activity of Anti-BoNT Antibodies

Antibodies of the present disclosure, individually or in combination, can inhibit (reduce or eliminate) the catalytic activity of botulinum neurotoxin (e.g. Type A). This inhibitory activity can be evaluated in vivo or in vitro. In vivo inhibition measurements simply involve measuring changes in the inhibitory concentration (e.g., IC₅₀ or other standard metric) due to a BoNT neurotoxin administration due to the presence of one or more antibodies being tested for inhibitory activity. An example of an in vitro experiment involves using a substrate of BoNT that releases a detectable signal when cleaved by the BoNT/A Lc. Details may be found in the Examples section below. In vivo, the neurotoxin can be directly administered to the test organism (e.g. mouse) or the organism can harbor a botulism infection (e.g., be infected with Clostridium botulinum). The antibody can be administered before, during, or after the injection of BoNT neurotoxin or infection of the test animal. A decrease in the rate of progression, or mortality rate indicates that the antibody(ies) have inhibitory activity.

Antibodies of the present disclosure, individually or in combination, may also reduce toxicity of botulinum neurotoxin. This activity can be evaluated in vivo or in vitro. In vivo measurements of toxin inhibition can involve measuring changes in the lethality (e.g., LD₅₀ or other standard metric) due to a BoNT neurotoxin administration in the presence of one or more antibodies being tested for inhibitory activity. The neurotoxin can be directly administered to the test organism (e.g. mouse) or the organism can harbor a botulism infection (e.g., be infected with Clostridium botulinum). The antibody can be administered before, during, or after the injection of BoNT neurotoxin or infection of the test animal. A decrease in the rate of progression, or mortality rate indicates that the antibody(ies) have inhibitory activity.

Examples of methods to assess the ability of an antibody to inhibit BoNT activity in vitro are described in the Examples section below. One example of an in vitro assay for inhibitory activity uses a hemidiaphragm preparation (Deshpande et al. (1995) Toxicon 33: 551-557). Briefly, left and right phrenic nerve hemidiaphragm preparations are suspended in physiological solution and maintained at a constant temperature (e.g. 36° C.). The phrenic nerves are stimulated supramaximally (e.g. at 0.05 Hz with square waves of 0.2 ms duration). Isometric twitch tension is measured with a force displacement transducer (e.g., GrassModel FT03) connected to a chart recorder. Antibodies are then added either with or after contacting the nerve preparations with BoNT (e.g. BoNT/A1, BoNT/A2, BoNT/B1, etc.). The time to 50% twitch tension reduction can be determined (e.g., three times for BoNT alone and three times for antibody plus BoNT). Differences between times to a given (arbitrary) percentage (e.g. 50%) twitch reduction can be determined by standard statistical analyses (e.g. two-tailed t test) at standard levels of significance (e.g., a P value of <0.05 considered significant).

### Compositions

The anti-BoNT antibodies of the present disclosure find use in treating a subject (e.g., a human) exposed to BoNT, and includes treatment during a stage in the disease where the toxin has entered the neuron of the subject (e.g., to provide for reversal of intoxication). Typically compositions comprising one, two, or more different antibodies can be provided as a pharmaceutical composition and administered to a mammal (e.g., to a human) in need thereof.

Compositions contemplated herein may contain one, two, three, or more different antibodies selected from the following: Aa1, A26, A3, A16, A23, A10, Aa12, Aa6, Aa9, A8, A21, A19, Aa8, Aa5, Aa11, A8.1a, B01, B04, B12, B22, Bc1, Bc2, Bc3, Bc4, Bc5, Bc6, Bc7, Bc8, Bc9, Bc10, Bc11, Bc12, Bc13, or Bc14. The composition may optionally further include antibodies comprising one or more CDRs from these antibodies, and/or one or more antibodies comprising mutants or derivatives of these antibodies.

The antibodies of the present disclosure can be used individually, and/or in combination with each other, and/or in combination with other known anti-BoNT antibodies (see, e.g., U.S. Pat. No. 7,563,874, US Pat Pub. No. 20080124328, PCT Pub No. WO/2009/008916, and PCT Application No. PCT/US09/52314., which are incorporated herein by reference for all purposes). These antibodies can be used individually, and/or in combination with each other, and/or in combination with other known anti-BoNT antibodies to form bispecific or polyspecific antibodies.

The subject composition encompasses compositions that specifically inhibit the catalytic activity of a serotype, such as serotype BoNT/A. For example, the composition may contain any combination of antibodies described above that specifically inhibit the cleavage activity of an Lc fragment.

An example of a composition of the present disclosure may include any of the combinations described above or one or more of the antibodies disclosed in FIG. 7.

Where combinations of antibodies are disclosed herein, such combinations can be provided in a single formulation or can be provided as separate formulations in a kit, where the separate formulations may contain a single antibody or two antibodies. Such separate formulations of a kit may be combined prior to administration or administered by separate injection.

The BoNT-inhibitory antibodies provided by the present disclosure are useful for parenteral administration, and may further find use in topical or oral administration. Administration may be systemic or local. The pharmaceutical compositions can be administered in a variety of unit dosage forms depending upon the method of administration. For example, unit dosage forms suitable for oral administration include powder, tablets, pills, capsules and lozenges. The antibodies comprising the pharmaceutical compositions of the present disclosure, when administered orally, are preferably protected from digestion. This is typically accomplished either by complexing the antibodies with a composition to render them resistant to acidic and enzymatic hydrolysis or by packaging the antibodies in an appropriately resistant carrier such as a liposome. Means of protecting proteins from digestion are well known in the art.

The pharmaceutical compositions of the present disclosure are particularly useful for parenteral administration, such as intravenous administration, intramuscular, subcutaneous, or into a body cavity or lumen of an organ. The compositions for administration will commonly comprise a solution of one or more BoNT-inhibitory antibody dissolved in a pharmaceutically acceptable carrier, which may be an aqueous carrier. A variety of aqueous carriers can be used, e.g., buffered saline and the like.

Non-aqueous pharmaceutically acceptable carriers (excipients) are known to those of skill in the art. Such excipients can comprise any substance that is biocompatible and liquid or soft enough at the subject's body temperature to release the active agent(s) (e.g., antibodies) somatotropin into the subject's bloodstream at a desired rate. Non-aqueous carriers are usually hydrophobic and commonly organic, e. g., an oil or fat of vegetable, animal, mineral or synthetic origin or derivation.

The various solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of BoNT-inhibitory antibody in these formulations can vary widely, and will be selected primarily based on fluid volumes, viscosities, body weight and the like in accordance with the particular mode of administration selected and the patient's needs.

Thus, a typical pharmaceutical composition for intravenous administration would be about 0.1 to 10 mg per patient per day. Dosages from about 1 mg up to about 200 mg per patient per day can be used. Methods for preparing parenterally administrable compositions will be known or apparent to those skilled in the art and are described in more detail in such publications as Remington's Pharmaceutical Science, 15th ed., Mack Publishing Company, Easton, Pa. (1980).

The compositions containing the BoNT-inhibitory antibodies of the present disclosure or a cocktail thereof are generally administered for therapeutic treatments. Preferred pharmaceutical compositions are administered in a dosage sufficient to inhibit (mitigate or eliminate) the catalytic activity of BoNT toxin(s) (i.e., reduce or eliminate a symptom of BoNT poisoning (botulism)). An amount adequate to accomplish this is defined as a “therapeutically effective dose.” Amounts effective for this use will depend upon the severity of the disease and the general state of the patient's health.

Single or multiple administrations of the compositions may be administered depending on the dosage and frequency as required and tolerated by the patient. In any event, the composition should provide a sufficient quantity of the antibodies of the present disclosure to effectively treat the patient.

### Methods of Use

The antibodies of the present disclosure find use in the treatment of a subject exposed to a BoNT. Accordingly, the present disclosure provides methods of treating a subject by administering a therapeutically effective amount of an anti-BoNT antibody disclosed herein to a subject exposed to a botulinum neurotoxin. The methods can involve administering an effective amount of such an anti-BoNT antibody to the subject so as to provide for inhibition of activity of botulinum neurotoxin in the subject. Such methods include treatment of a subject that suffers from intoxication by botulinum neurotoxin. Methods of the present disclosure include those that provide for administering an anti-BoNT antibody as disclosed herein in an amount effective to reverse BoNT-induced paralysis in a subject.

“Therapeutically effective amount” means an amount of antibody or antibody fragment that produces the effects for which it is administered. The exact dose will be ascertainable by one skilled in the art. As known in the art, adjustments based on age, body weight, sex, diet, time of administration, drug interaction and severity of condition may be necessary and will be ascertainable with routine experimentation by those skilled in the art. A therapeutically effective amount is also one in which the therapeutically beneficial effects outweigh any toxic or detrimental effects of the antibody or antibody fragment.

An exemplary, non-limiting range for a therapeutically or prophylactically effective amount of an antibody or antibody portion of the invention is 0.1-20 mg/kg, more preferably 1-10 mg/kg.

### Kits

Kits for the treatment of botulism are also provided. Kits will typically comprise one or more anti-BoNT antibodies (e.g., BoNT-inhibitory antibodies for pharmaceutical use). For diagnostic purposes, the antibody(s) can optionally be labeled. In addition the kits will typically include instructional materials disclosing means of use BoNT-inhibitory antibodies in the treatment of symptoms of botulism. The kits may also include additional components to facilitate the particular application for which the kit is designed. The kits may additionally include buffers and other reagents routinely used for the practice of a particular method. Such kits and appropriate contents are well known to those of skill in the art.

Kits provided for the treatment of botulism may contain one or more BoNT inhibitory antibodies. The antibodies can be provided separately or mixed together. Typically the antibodies will be provided in a sterile pharmacologically acceptable excipient. The antibodies can also be provided pre-loaded into a delivery device (e.g., a disposable syringe).

The kits can optionally include instructional materials teaching the use of the antibodies, recommended dosages, contraindications, and the like.

### EXAMPLES

The following examples are offered to illustrate, but not to limit any embodiments provided by the present disclosure.

The following methods and materials were used in the present example.

Methods and materials

Oligonucleotides for library construction:
Leader-L01:
(SEQ ID NO: 36)
5′-GTCCTGGCTGCTCTTCTACAAGG-3′
CH2-L01:
(SEQ ID NO: 37)
5′-ATGGAGAGGACGTCCTTGGGT-3′
GAP5-V_{H}H back:
(SEQ ID NO: 38)
5′-CCTGACTATGCAGCTAGCGGTGCCATGGCACAGGTGCAGCTCGTGGAGTCTGGGG-3′
(SEQ ID NO: 39)
5′-CCTGACTATGCAGCTAGCGGTGCCATGGCACAGGTACAGCTGGTGGAGTCTGGGG-3′
(SEQ ID NO: 40)
5′-CCTGACTATGCAGCTAGCGGTGCCATGGCAGAGGTGCAGCTGGTGGAGTCTGGGG-3′
(SEQ ID NO: 41)
5′-CCTGACTATGCAGCTAGCGGTGCCATGGCAGATGTGCAGCTGGTGGAGTCTGGGG-3′
(SEQ ID NO: 42)
5′-CCTGACTATGCAGCTAGCGGTGCCATGGCAGCGGTGCAGCTGGTGGAGTCTGGGG-3′
(SEQ ID NO: 43)
5′-CCTGACTATGCAGCTAGCGGTGCCATGGCAGCCGTGCAGCTGGTGGATTCTGGGG-3′
(SEQ ID NO: 44)
5′-CCTGACTATGCAGCTAGCGGTGCCATGGCACAGGTGCAGCTGGTGGAGTCTGTGG-3′
(SEQ ID NO: 45)
5′-CTGACTATGCAGCTAGCGGTGCCATGGCACAGGTAAAGCTGGAGGAGTCTGGGG-3′

Underlined sequence anneals upstream of NcoI site in vector pYD2 for cloning by gap repair.

GAP3-V_{H}H forward:
(SEQ ID NO: 46)
5′-CTTACCTTCGAAGGGCCCGCCTGCGGCCGCTGATGAGACAGTGACCA
GGGTSCCCTG-3′
5′-CTTACCTTCGAAGGGCCCGCCTGCGGCCGCTGAGGAGACGGTGACCT
GGGTCCCCTG-3′

Underlined sequence anneals downstream of NotI site in vector pYD2 for cloning by gap repair.

Strains, media antibodies, and toxins. Saccharomyces cerevisiae strain EBY100 (GAL1-AGA1TURA3 ura3-52 trp1 leu2Δ1 his3Δ200 pep4::HIS3 prb1Δ1.6R can1) was maintained in YPD medium (1% yeast extract, 2% peptone, 2% dextrose) (Current Protocols in Molecular Biology, John Wiley and Sons, Chapter 13.1.2). EBY100 transformed with expression vector pYD2 (Razai A et al. (2005) J Mol Biol 351:158-169) was selected on SD-CAA medium (0.7% yeast nitrogen base, 0.1M Sodium phosphate, 0.5% casamino acids, 2% dextrose, 0.006% Leucine). V_{H}H yeast surface display was induced by transferring yeast cultures from SD-CAA to SG-CAA medium (identical to SD-CAA medium except the glucose was replaced by galactose) and grown at 18° C. for 24˜48 hr as described previously (Feldhaus M J et al. (2003) Nat Biotechnol 21:163-170). Bacteria strain E. coli DH5α, was used for cloning and preparation of plasmid DNA. Pure BoNT/A1 (Hall hyper) was purchased from Metabiologics (Madison, Wis.). Mouse anti-SV5 antibody was purified from hybridoma supernatant using Protein G and directly labeled with Alexa-647 using a kit provided by the manufacturer (Molecular Probes). Recombinant human BoNT/A antibodies 3D12 and AR2 were purified from Chinese hamster ovary cells (CHO) supernatants (Razai, A. et al. (2005) J Mol Biol 351:158-169; Nowakowski, A et al. (2002) Proc Natl Acad Sci USA 99:11346-11350)

Expression and purification of proteins: BoNT/A LC448 was amplified from a synthetic BoNT/A1 gene (Levy R et al. (2007) J Mol Biol 365:196-210), subcloned into an IPTG inducible pET15b vector, and expressed in BL21 (DE3) cells at 18° C. overnight. The cells were broken with Bugbuster Master (Novagen), and hexahistidine tagged BoNT/A Lc448 was purified by immobilized metal affinity chromatography (IMAC) using Ni-NTA agarose (Qiagen) followed by ion exchange chromatography. BoNT/A Lc425, residues 1-425) was constructed and expressed similarly. BoNT/A Lc₄₂₅ was purified by IMAC and the BoNT/A Lc₄₂₅-Aa1 V_{H}H complex was further purified by size exclusion chromatography on a Superdex200 column (GE healthcare). The SNAP25₁₄₁₋₂₀₆-pGEX-2T construct, which contains a fragment of human SNAP25, was a gift from Dr. Joseph Barbieri (Medical College of Wisconsin, Milwaukee, Wis.) (Chen S et al. (2006) J Biol Chem 281:10906-10911) and was transformed into BL21 (DE3) cells and expression induced with 0.5 mM IPTG at 18° C. overnight. GST-SNAP25 was purified on a glutathione-sepharose 4B column (Clontech).

Naïve llama V_{H}H library construction: Total RNA was isolated from 40 ml fresh blood from three llamas using PAX gene Blood RNA kit (PreAnalytiX, QIAGEN/BD). The cDNA was synthesized by RT-PCR using a ThermoScript RT-PCR Kit (Invitrogen), with oligo dT and random hexamer as primers. The gene fragment encoding the heavy chain variable domains was PCR amplified with primers annealing at the leader sequence and at the CH2 exon of the llama heavy chains, (leader-L01 and CH2-L01; see Oligonucleotides for library construction in the Supplemental Materials for the sequences). The design of leader-L01 was based on llama V_{H}H germline genes AF305944, AF305945, AF305946, AF305947, and AF305948 (Genbank). The sequence of CH2-L01 was derived from llama germline constant region genes AF132604 and AF132605 (Woolven B P et al. (1999) Immunogenetics 50:98-101). The PCR product showed two bands, ˜550 bp and 800 bp, on an agarose gel and the 550 bp fragment was (V_{H}H-CH2 without CH1) gel extracted and reamplified with primers GAP5-V_{H}HBack and GAP2-VHHForward. For cloning by gap repair (Orr-Weaver T L et al. (1983) Proc Natl Acad Sci USA 80:4417-4421), 29 (V_{H}H Forward) or 30 (V_{H}H Back) nucleotides complementary to the 3′ or 5′ ends of Nco1-Not1 digested pYD2 were appended to the primers. Approximately, 22 μg of V_{H}H gene and 65 pg of NcoI-NotI-digested pYD2 vector was used to transform lithium acetate treated EBY cells (Gietz R. D et al. (2007) Nat Protoc 2:38-41). Transformation mixtures were cultured and subcultured in SD-CAA. Library size was determined by plating serial dilutions of the transformation mixture on SD-CAA plates. V_{H}H display was induced by culturing in SG-CAA media for more than 12 hr at 18° C.

Selection of BoNT/A Lc binding V_{H}H: An amount of yeast at least ten times larger than the library size or the sort output from the previous round were used for each selection. All the washing and staining were done with FACS buffer (phosphate-buffered saline (pH 7.4), 0.5% bovine serum albumin). For flow sorting, 200 nM of BoNT/A Lc₄₄₈ was used for the first two rounds, and 100 nM was used for the third round. The volume of the incubation was chosen to ensure that BoNT/A Lc448 was in at least a fivefold excess over the number of V_{H}H (assuming 5×10⁵ V_{H}H/yeast), and the incubation times were 30 min, long enough for the reaction to achieve equilibrium. To minimize dissociation, all the following washing and staining steps were performed at 4° C. using ice-cold FACS buffer. After incubation with BoNT/A Lc448 and washing in FACS buffer, the yeast sample was incubated with the human BoNT/A Lc448-specific IgG1 monoclonal antibodies (mAb) ING2 and 5A20.4 (Levy R et al. (2007) J Mol Biol 365:196-210) which bind non-overlapping epitopes (1 μg/ml), followed by incubation with 1 μg/ml PE-labeled goat anti-human Fc antibody (Jackson Immunogenetic) and 1 μg/ml Alexa-647-labelled anti-SV5 mAb. Cells were washed once and sorted on a FACSAria II. The V_{H}H expressing (SV5 positive) and BoNT/A Lc₄₈₈ binding population were gated for collection. Collected cells were grown in SD-CAA media and used for the next round of sorting after induction in SG-CAA. A yeast sample from the third round of sorting was plated on SD-CAA plates, and colonies on the plates were picked and grown in 96 deep-well plates. The individual clones were then induced for expression and screened for BoNT/A Lc₄₄₈ binding and binding clones were identified by DNA sequencing.

Expression and purification of V_{H}H: The genes of the selected clones that bound to BoNT/A LC448 were subcloned into the expression vector pSYN1 (Schier R et al. (1995) Immunotechnology 1:73-81), using the restriction enzymes NcoI or SfiI and NotI. The plasmid constructs were transformed into E. coli TG1 cells. Expression of V_{H}H was performed in shaker flasks by growing the bacteria in 2× YT. Expression was induced by adding 0.2 mM IPTG and growing at 18° C. overnight. After pelleting the cells, the periplasmic proteins were extracted by osmotic shock. Hexahistidine tagged V_{H}H proteins were purified by IMAC on a Ni-NTA agarose column as described by Schier R et al. (1995) Immunotechnology 1:73-81. To increase the expression level for crystal growing, the Aa1 clone was subcloned into pET20b (Novagen), and transformed into Rosetta (DE3) (Novagen). The expression and purification were performed in the same way as above.

Measurement of yeast-displayed V_{H}H affinity for BoNT/A Lc: The equilibrium dissociation constant (K_{D}) of yeast displayed V_{H}H was measured by flow cytometry as previously described for yeast displayed single chain Fv (scFv) (Razai, A. et al. (2005) J Mol Biol 351:158-169; Garcia-Rodriguez C et al. (2007) Nat Biotechnol 25:107-116). Briefly 105 SG-CAA-induced yeast displaying V_{H}H were incubated with one of six different BoNT/A Lc₄₄₈ concentrations spanning the expected K_{D} from 0 nM to a value providing saturated binding. Incubation times and volumes were chosen to ensure that BoNT/A Lc₄₄₈ was in at least fivefold excess over the number of V_{H}H and the equilibrium was reached. BoNT/A Lc₄₄₈ binding was detected by incubating with mAbs ING2 and 5A20.4 followed by PE-anti-human Fc and Alexa647-SV5 mAb. Fluorescence signal was measured on a LSRII flow cytometer (BD Biosciences). The V_{H}H displaying population was gated and the mean fluorescence intensity for BoNT/A LC₄₄₈ binding recorded. To determine K_{D}, the fluorescence and BoNT/A Lc₄₄₈ concentration data was then fit into equation:

F=F_{back}+Fₘₐₓ[L]/(K_{D}+[L])

where F_{back} is the fluorescence intensity when there is no BoNT/A Lc₄₄₈, Fₘₐₓ is the fluorescence intensity when binding is saturated, and [L] is the concentration of BoNT/A LC₄₄₈.

Measurement of the Aa1 V_{H}H solution phase affinity at equilibrium and binding kinetics: The Aa1 V_{H}H solution K_{D} for BoNT/A Lc₄₄₈ was measured by flow fluorimetry in a KinExA instrument as previously described for IgG (Razai, A. et al. (2005) J Mol Biol 351:158-169; Garcia-Rodriguez C et al. (2007) Nat Biotechnol 25:107-116). Briefly, Aa1 V_{H}H was serially diluted into a constant concentration of BoNT/A Lc₄₄₈, where the Aa1 concentration was varied from less than 0.1 to greater than tenfold above the value of the apparent K_{D}. The BoNT/A Lc₄₄₈ concentration was no more than fourfold above the K_{D} to ensure a K_{D} controlled measurement. Samples were allowed to reach equilibrium for as long as two days, then each of the reactions were passed over a flow cell with a 4 mm column of Azlactone beads (Sapidyne Instruments) covalently coated with Aa1 V_{H}H to capture the free BoNT/A Lc₄₄₈. The amount of BoNT/A Lc₄₄₈ bound to the beads was quantitated by flowing Alexa-647 labeled BoNT/A Lc₄₄₈ mAb 5A20.4 over the beads. The equilibrium titration data were fit to a 1:1 reversible binding model using KinExA Pro Software to determine the K_{D.} To measure the association rate constant (kₒₙ), approximately 1-2 ml of Aa1 V_{H}H and BoNT/A Lc₄₄₈ reaction mixture was passed over a freshly packed flow cell with Aa1 V_{H}H coated beads. The amount of BoNT/A Lc₄₄₈ bound to the beads was quantitated as described above. No less than ten time points were sampled with the internal time approximately 500 sec. The exponential decrease in the concentration of free BoNT/A Lc₄₄₈ as a function of time was fit to a standard bimolecular rate equation using the KinExA Pro Software to determine the koₙ (Drake A W et al. (2004) Anal Biochem 328: 35-43). The dissociation rate constant (koff) was calculated as K_{D}×kₒₙ.

Cleavage of SNAP25 by BoNT/A Lc₄₄₈: BoNT/A Lc448 was mixed with or without V_{H}H proteins in 50 mM Tris buffer, pH 8.0. GST⁻SNAP25₁₄₁₋₂₀₆ was added into the mixture to initiate the reaction. The final volume was 40 μl, and final concentration was approximately 20 nM for BoNT/A Lc₄₄₈ and 5 μM for GST-SNAP25₁₄₁₋₂₀₆. The concentration of each VHH was at least 50 fold higher than the BoNT/A Lc₄₄₈ concentration. The reaction was run at room temperature for 10 min or for the indicated time, stopped by adding SDS-PAGE loading buffer and heated for 10 min at 99° C., then analyzed by SDS-PAGE. The gel was stained in 0.1% Coomassie Blue R-250.

IC₅₀ measurement: The Aa1 50% inhibitory concentration (IC₅₀) for BoNT/A Lc₄₄₈ was determined by using a fluorescent resonance energy transfer (FRET) based cleavage assay with YsCsY as substrate (Pires-Alves M et al. (2009) Toxicon 53:392-399). Reactions were performed in black 96-well plates (Corning) in assay buffer (10 mM HEPES, 150 mM K glutamate, 0.01% Tween20, pH 7.2) (modified from Vaidyanathan V V et al. (1999) J Neurochem 72:327-337)). YsCsY was mixed with two-fold serially diluted Aa1 V_{H}H, with the estimated IC₅₀ equal to the median V_{H}H concentration. After pre-incubation at 30° C. for 15 min in a microplate fluorescence reader (Spectra Max Gemini, Molecular Devices), BoNT/A Lc₄₄₈ was added to the wells by multi-channel pipet, to initiate the reaction. The total reaction volume was 100 μl, the final concentration was 0.5 μM for YsCsY and 400 pM for BoNT/A Lc₄₄₈. Fluorescence was measured in the monochromatic mode with excitation at 425 nm and emission at 525 nm. Initial rates were determined from the change in YFP fluorescence reading (525 nm); the fluorescent data from the first 40 sec was fit to a simple linear regression model Y=RX+C (where Y=YFP fluorescence, R=slope, X=time, and C=y-intercept) and the “−R” value was taken as the initial rate (the R value was negative since the signal at 525 nm gets weaker with substrate cleavage). The Aa1 IC₅₀ was determined by fitting the initial rate and log Aa1 concentration to a sigmoidal dose-response (variable slope) model (GraphPad Prism).

CD spectroscopy: All circular dichroism (CD) experiments were performed on an Aviv model 202SF circular dichroism spectrometer with a 0.2 cm path length cell containing 18-20 mM Aa1 V_{H}H protein in PBS buffer. Wavelength data were collected in 1 nm steps with an averaging time of 15 sec. Thermal denaturation/refolding experiments were run from 10 to 90° C. in 2.5 degree steps, at a two-degree/minute rate of change with 1.5 min equilibration and 30 sec data averaging at each temperature. Tm values were obtained from the minima of the first derivative of θ versus T plots.

Crystallization and Structure Determination: The BoNT/A Lc₄₂₅-Aa1 V_{H}H complex was formed by mixing at a 1:1.5 molar ratio followed by purification using size exclusion chromatography on a Superdex 200 column (GE healthcare), with running buffer of 100 mM NaCl and 10 mM HEPES (pH 7.0). The complex eluate was then concentrated to 13 mg/mL by centrifugal ultrafiltration (Amicon). Crystals were grown by sitting nanodrop vapor diffusion at 20° C. using 13 mg/ml total protein sample and the Innovadyne Screenmaker crystallization robot. Crystals grew in 4 days with a precipitant/well solution containing 25% ethylene glycol and were flash frozen in liquid nitrogen directly from the sitting drop. Diffraction data were collected at the Advanced Photon Source GM/CA CAT beamline 23ID-B (Argonne, Chicago Ill.). Reflections were processed using HKL2000 (Otwinowski Z. et al. (1997) Methods in Enzymology 276: 307-326) and the initial structure solution was obtained by molecular replacement with Phaser (McCoy A J et al. (2007) Journal of applied crystallography 40:658-674) using the light chain from PDB code 3BTA (residues 2:425) and a polyalanine model of the llama V_{H}H domain from PDB code 1I3V as search models. Cycles of refinement and manual rebuilding were performed with CNS (Brunger A T et al. (1998) Acta crystallographica 54:905-921) and Coot, respectively (Emsley, P., and Cowtan, K. (2004) Acta crystallographica 60:2126-2132). Residue Ala248 at position P2 of the hydrolysed peptide bond is the only Ramachandran outlier (Procheck), although the electron density for this region is well defined. Crystallographic details are listed in Table 2 below and structure figures were generated with Pymol Molecular Graphics System (pymol.org).

TABLE 2
Summary of Crystallographic Data

| | BoNT/A Lc425/Aa1 V_{H}H |
|---|---|
| PBD Code | T.B.D. |
| Space group | C2 |
| Unit Cell (Å) | a = 168.2, b = 48.8, c = 103.6 |
| X-ray Source | APS GM/CA, 1.03 Å λ |
| Resolution Limits (Å) | 40.0-2.6 (2.64-2.60) |
| Reflections | |
| Total Collected | 64,075 |
| Unique | 22,641 |
| Redundancy | 2.8 (1.9) |
| I/σ | 7.3 (2.2) |
| Completeness (%) | 96.5 (83.9) |
| R_{sym} (%) | 11.7 (31.4) |
| Refinement | |
| Resolution Range (Å) | 40.0-2.6 |
| R-work (%) | 22.1 |
| R-free (%) | 26.2 |
| Ramachandran statistics | |
| Favored | 386 |
| Allowed | 84 |
| Generous | 3 |
| Disfavored | 1 |

Data in parentheses are for the highest resolution shell. R-free was computed using 5% of the data. Ramachandran statics do not include Gly and Pro residues.

### Example 1

### Generation and Initial Characterization of Single Domain Antibodies to Botulinum Neurotoxin Type A Light Chain.

To generate a panel of single domain antibodies binding the BoNT/A Lc, a non-immune llama single domain library was constructed for display on the surface of Saccharomyces cerevisiae. Briefly, whole blood was isolated from llamas without prior immunization and RNA prepared. After first strand cDNA synthesis, llama specific primers annealing to the VH and V_{H}H leader sequence genes and to the CH2 gene were used to PCR amplify the VH and V_{H}H gene repertoires. V_{H}H repertoires were separated from VH repertoires by running the PCR fragments on a gel and excising the smaller band. The V_{H}H gene repertoire was reamplified and cloned into the vector pYD2 for display as a C-terminal fusion to the AgaII protein on the surface of Saccharomyces cerevisiae. After transformation, a library of size of 6.1×10⁷ transformants with a V_{H}H sized insert was obtained. DNA sequencing of 50 V_{H}H genes picked at random revealed 44 unique sequences, indicating that the library was diverse.

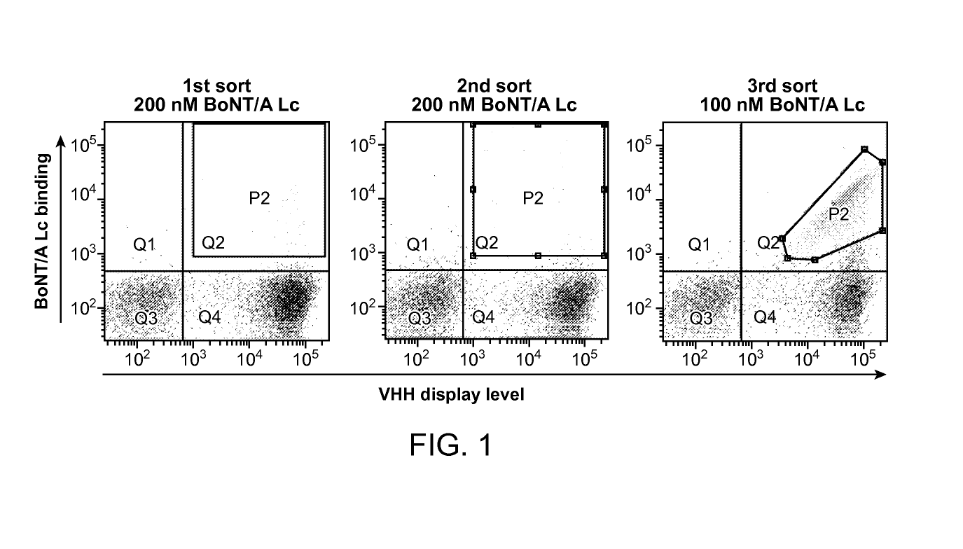

To generate BoNT/A Lc specific single domain antibodies, surface display was induced and the yeast incubated with recombinant BoNT/A Lc. After staining with anti-BoNT/A Lc mAbs and a mAb directed to the C-terminal SV5 epitope tag, all yeast displaying V_{H}H and bound to BoNT/A Lc were flow sorted and collected (FIG. 1). After amplification by growth in liquid culture, surface display was induced and the staining, sorting, and growth cycle repeated twice more (FIG. 1). After three rounds of sorting, yeast were plated and 48 individual colonies analyzed for binding to BoNT/A Lc. The V_{H}H gene of each binding clone was sequenced revealing the presence of 15 unique V_{H}H, two pairs of which (Aa1 and A23; Aa12 and A10) were clonally related based on the V_{H}H complementarity determining region 3 (CDR3) sequence (Table 1). The affinity of each of the yeast displayed V_{H}H for BoNT/A Lc was determined by flow cytometry and found to range from a low of 230 nM (clone A8) to a high of 30 pM (clone Aa1) with an average K_{D} of 5.6×10⁻⁸ M (Table 1).

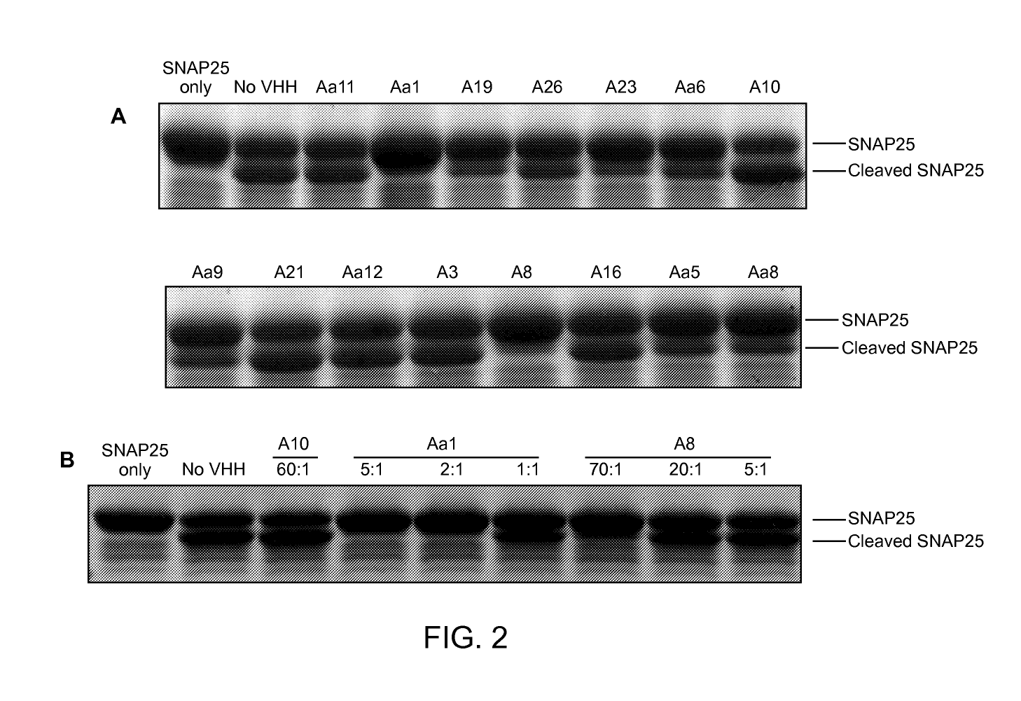

To determine whether any of the V_{H}H inhibited the cleavage of SNAP25 by BoNT/A Lc, each V_{H}H was subcloned for expression in E. coli and purified by IMAC to greater than 90% purity. Each purified V_{H}H was then evaluated for its ability to prevent the cleavage of a GST-SNAP25₁₄₁₋₂₀₆ fusion protein as determined by SDS-PAGE (FIG. 2a). Eight of fifteen clones (Aa1, A19, A23, Aa6, Aa9, A8, Aa5, and Aa8) showed partial or complete inhibition of cleavage (FIG. 2A). Higher affinity V_{H}H inhibited cleavage at lower molar ratios to BoNT/A Lc compared to lower affinity V_{H}H (FIG. 2B).

### Example 2

### Further Characterization of the Aa1 V_{H}H Fragment

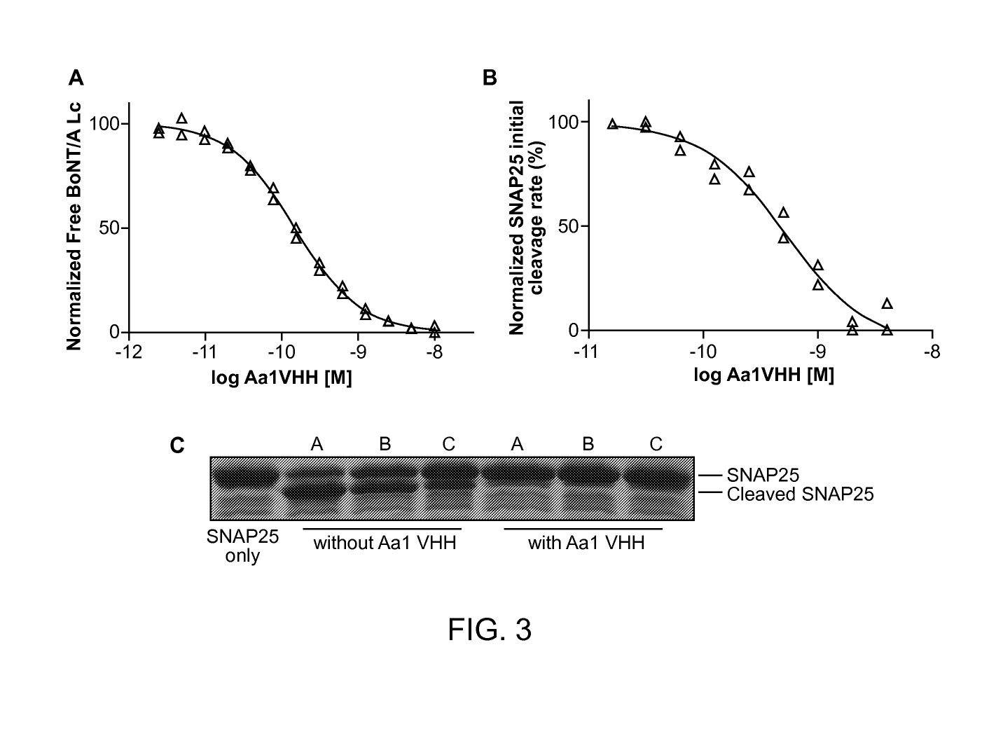

Aa1 V_{H}H binding affinity and potency of BoNT/A Lc inhibition. The solution K_{D} and binding kinetics of the Aa1 V_{H}H were determined by flow fluorimetry in a KinExA instrument (FIG. 3A). The K_{D} was measured to be 1.47×10⁻¹⁰ M, with a kₒₙ as 4.39×10⁷ M⁻¹s⁻¹ and a k_{off} calculated to be 6.66×10⁻³ s⁻¹. The V_{H}H solution K_{D} is within 5 fold of the K_{D} measured for the yeast displayed V_{H}H by flow cytometry. To determine the potency of BoNT/A Lc inhibition, the IC₅₀ of the Aa1 V_{H}H was measured by FRET using as a substrate CFP and YFP connected through SNAP25 residues 141-206 (Pires-Alves M . et al. (2009) Toxicon 53:392-399). The initial cleavage rate as a function of V_{H}H concentration was used to calculate the IC₅₀ which was determined to be 5.2×10⁻¹⁰ M (FIG. 3B).

Stability of the Aa1 V_{H}H fragment. The ability of the Aa1 V_{H}H to withstand the intracellular reducing environment was determined by incubating the Aa1 V_{H}H with either 20 mM glutathione or 14 mM β-mercaptoethanol at 37° C., followed by incubation with the BoNT/A Lc and GST-SNAP25₁₄₁₋₂₀₆. The Aa1 V_{H}H was still able to prevent BoNT/A Lc cleavage of SNAP25 after a15 min incubation with either reducing agent (FIG. 3C).

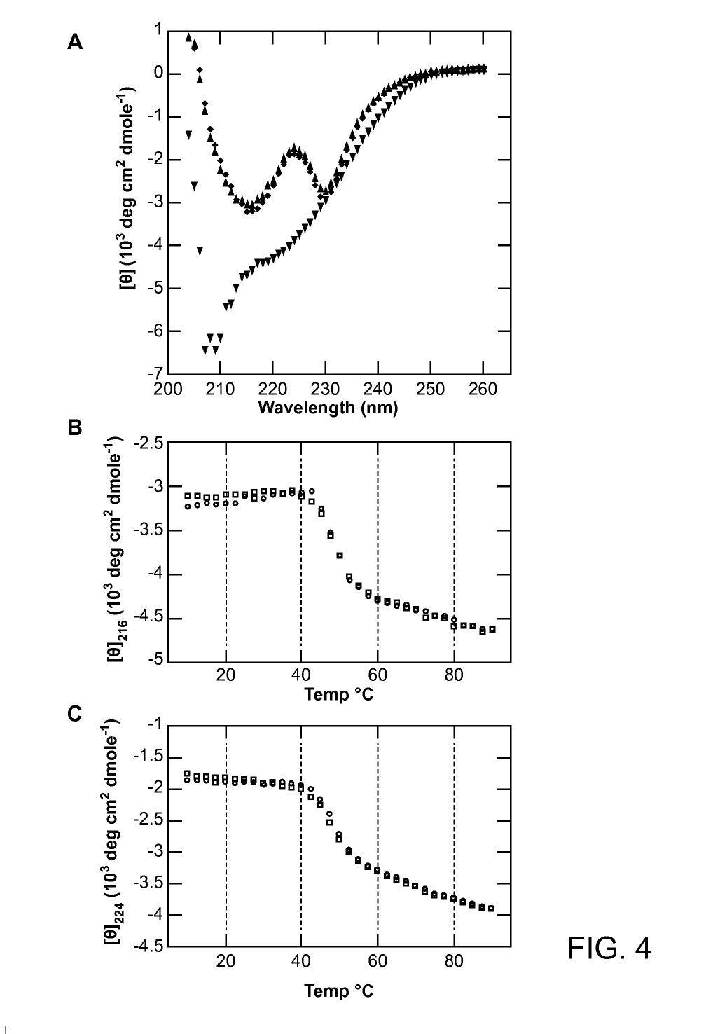

CD was utilized to test the thermostability of the Aa1 V_{H}H fragment. The far UV CD spectrum of the Aa1 protein displayed negative minima at 216 and 230 nm and a maximum at 224 nm (FIG. 4A). These features in the CD spectrum are consistent with the mixture of □-sheet and α-helical secondary structure observed in immunoglobulin domains. To investigate the melting temperature and potential reversibility of thermal denaturation, changes in the protein conformation were monitored by CD at wavelengths of 216 and 224 nm (FIGS. 4B and 4C). These melting profiles revealed two-state unfolding kinetics with Tₘs of ˜49° C. as determined by the minima of the first derivative of θ versus T plots. The protein sample incubated at 90° C. visually appeared identical to the protein at 10° C. and did not show any evidence of aggregation or precipitation. Wavelength spectra recorded at 90° C. looked significantly different from the spectrum at 10° C. and revealed a minimum at ˜208 nm. The differences in spectra obtained at 10° C. to 90° C. are consistent with a transition to mostly random coil with a small component of residual secondary structure. The Aa1 protein was then cooled slowly and allowed to refold (FIGS. 4B and 4C). The refolding profile coincided well with the temperature induced unfolding profile, and wavelength spectra recorded at 10° C. before and after the melting experiment were superimposable (FIG. 4A), indicating the protein refolded properly.

The CD melting experiment was also performed in the presence of 1 mM TCEP to explore the role of the single disulfide bond connecting both halves of the immunoglobulin β-sandwich. The wavelength spectrum recorded at 10° C. before any heating was essentially identical to the spectrum in the absence of TCEP (FIG. 8). The melting profile recorded at 216 nm was similar to the non-reduced profiles with the primary melting transition occurring ˜50° C., although the elipticity trend following this transition had a positive slope, unlike the non-reduced sample (FIG. 8 and FIGS. 4B and 4C). In this case, the refolding CD profile of the reduced Aa1 was very different from the melting profile, and the wavelength spectrum recorded at 10° C. after the melting experiment confirmed that the structure was significantly perturbed. These data suggest that the disulfide linkage within the single immunoglobulin domain is essential for the refolding process following thermal denaturation.

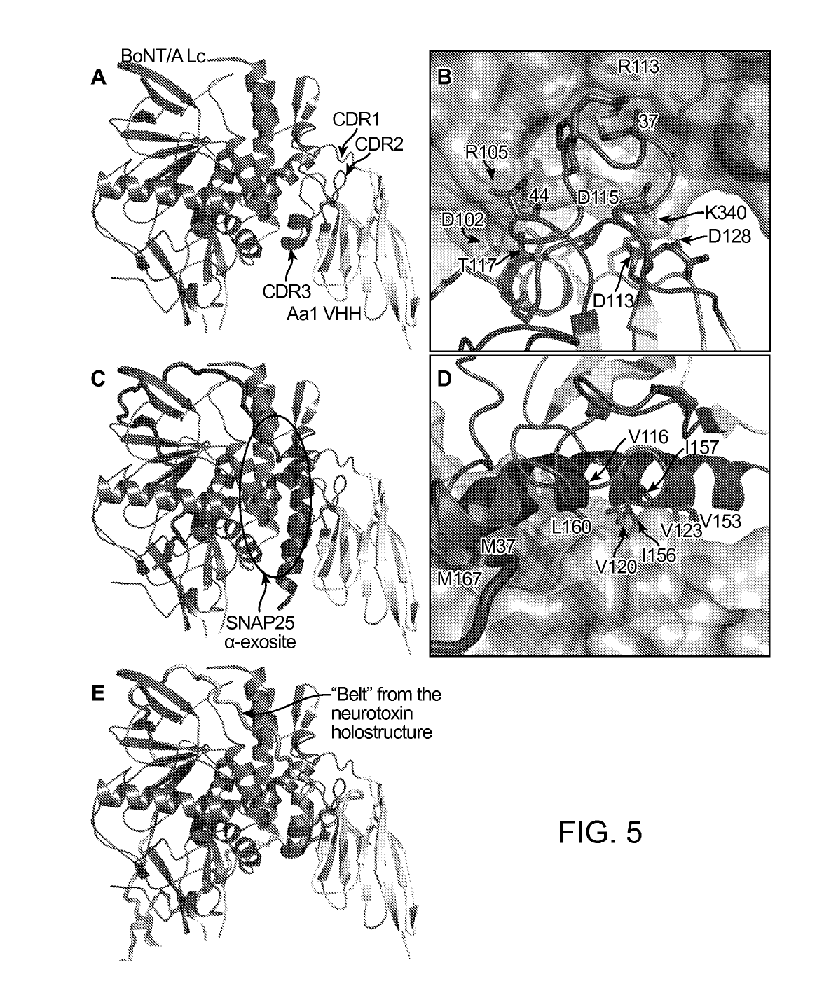

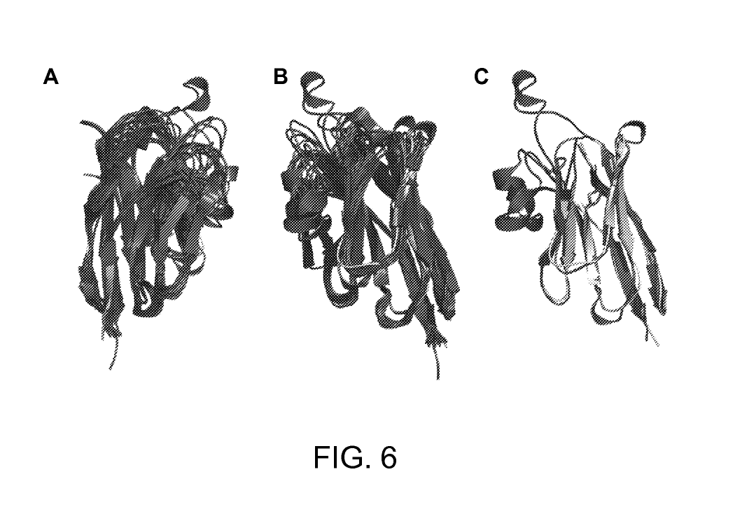

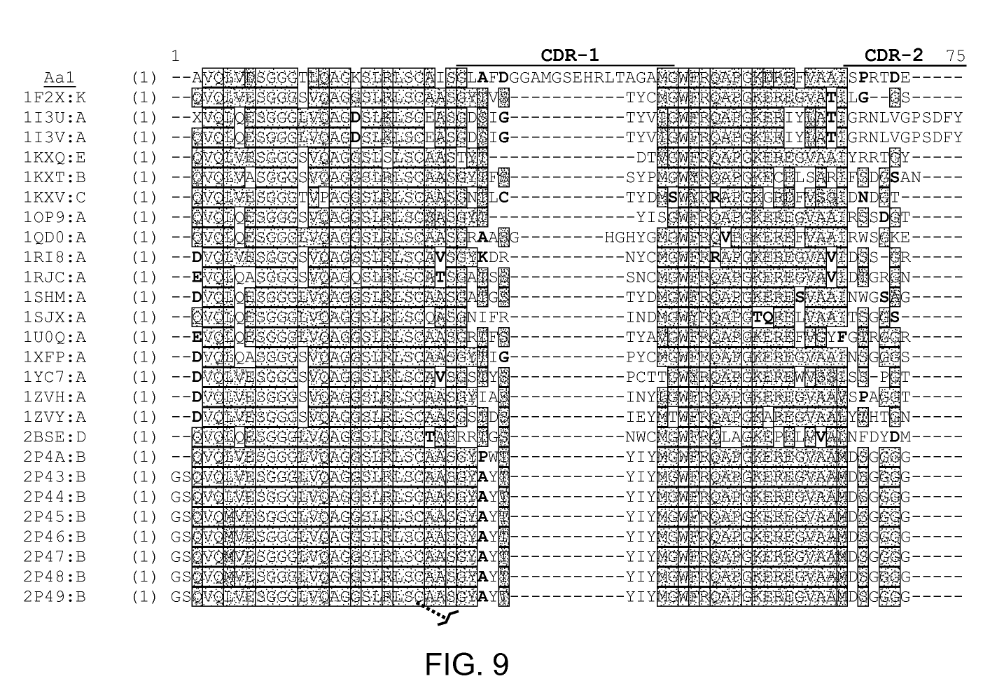

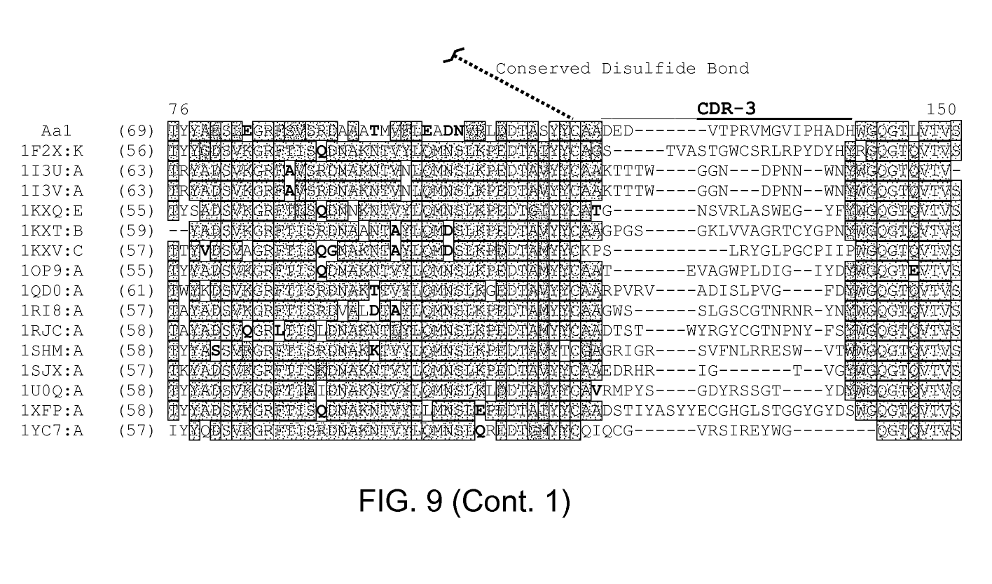

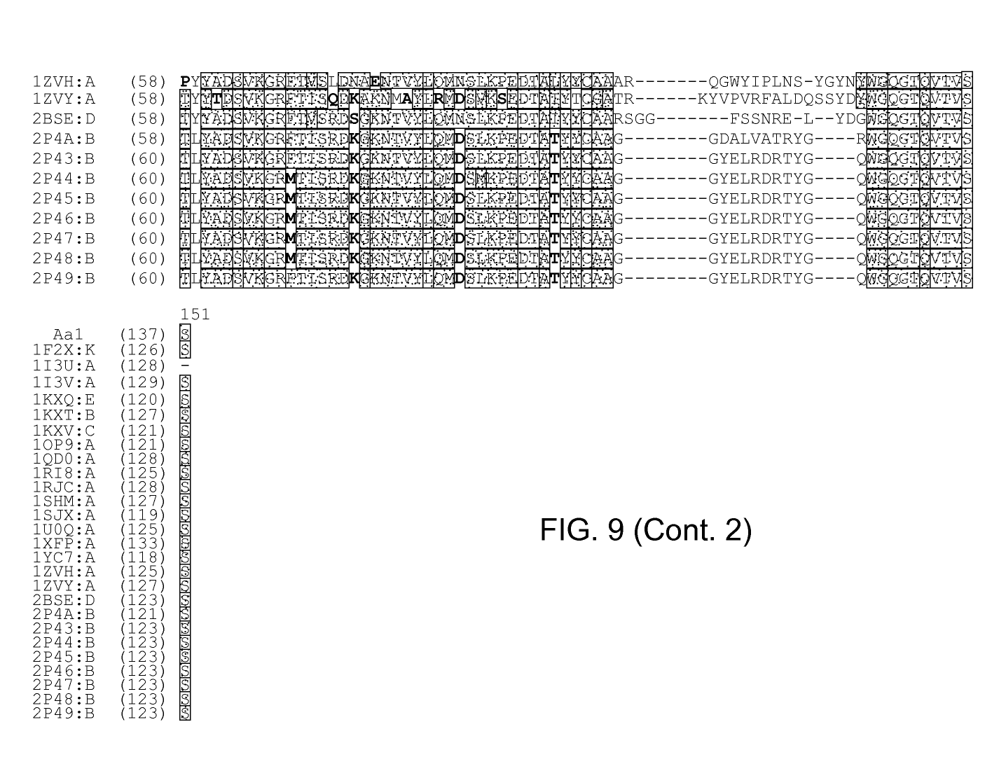

Structure of BoNT/A Lc-Aa1 V_{H}H complex. To understand the mechanism by which Aa1 inhibits BoNT/A Lc, the X-ray crystal structure of the BoNT/A Lc₄₂₅-Aa1 V_{H}H complex was determined at 2.6 Å resolution. The asymmetric unit contains a single BoNT/A Lc endopeptidase bound by the Aa1 V_{H}H fragment in a 1:1 stoichiometry. The V_{H}H fragment consists of a single immunoglobulin domain with three CDRs which were well defined by the experimental electron density. Binding by the llama antibody fragment is driven by CDR1 and CDR3 interactions resulting in the burial of ˜619 Å² of solvent exposed surface area from the interface of each protein (FIG. 5A; calculated by PISA) (Krissinel E et al. (2007) J Mol Biol 372:774-797). The structure of CDR1 appears to be unique as it is 11 amino acids larger than most V_{H}H structures (eight amino acids longer than the longest V_{H}H structure in the PDB) giving it a topology not seen in other V_{H}H structures (FIG. 6 and FIG. 9). A single sidechain interaction between Arg68 of CDR2 and a symmetry related endopeptidase molecule exists in the crystal lattice, but is likely to be a crystallization artifact and is unlikely to contribute to the observed binding affinity measured by FACS. The paratope formed by CDR1 and CDR3 is convex in shape facilitating interactions with a relatively concave epitope located on the opposite side of the endopeptidase active site. This interaction is stabilized by several hydrogen bonds (V_{H}H residues 117, 37, and 44 interactions with BoNT/A Lc residues 102,113,105, respectively) and salt bridges (Lc Lys340 interactions with V_{H}H Asp residues 113, 115, and 128) between Aa1 and the endopeptidase, and numerous van der Waals and hydrophobic interactions (FIG. 5B).

Superposition of this V_{H}H/endopeptidase structure with the structure of SNAP25 bound to the BoNT/A Lc enzyme (PDB code 1XTG; Breidenbach M A et al. (2004) Nature 432:925-929) reveals that the Aa1 paratope coincides with an alpha helical portion of the SNAP25 substrate (FIG. 5C). Both of these compete for a groove on the surface of the endopeptidase termed the α-exosite, which is occupied by the heavy chain belt of the neurotoxin holostructure (FIG. 5E; PDB code 3BTA) (Lacy D B et al. (1998) Nature Struct. Biol. 5:898-902). The CDR1 and CDR3 both form small alpha helices that appear to mimic the secondary structure of SNAP25 bound at the α-exosite. Although there is no primary sequence homology, the substrate mimicry is extended to tertiary structure because a number of hydrophobic residues found on the V_{H}H fragment coincide with similar residues found on SNAP25 (FIG. 5D). These residues include Met37 on CDR1 of the V_{H}H fragment which occupies the position of Met167 from SNAP25; Val116 from CDR3 which coincides with Leu160 of SNAP25; Val120 of CDR3 which occupies the space of Ile156 and Ile157 from SNAP25; and Val123 of CDR3 which replaces Val153 of the natural substrate. Because mutagenesis studies have indicated that the α-exosite plays a role in proper binding and cleavage of SNAP25 (Breidenbach M A et al. (2004) Nature 432:925-929; Rossetto O et al. (1994) Nature 372:415-416; Washbourne P et al. (1997) FEBS Lett 418:1-5), binding by Aa1 likely inhibits substrate cleavage by precluding efficient binding of SNAP25 and positioning of its scissile bond.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes. While the subject antibody, method, and composition have been particularly shown and described with references to preferred embodiments thereof, it will be understood by those skilled in the art that various changes in form and details may be made therein without departing from the scope of the invention encompassed by the appended claims.

1. An isolated antibody that specifically binds a Botulinum neurotoxin (BoNT) and inhibits the activity of BoNT in cleavage of its substrate.

2. The isolated antibody of claim 1, wherein said substrate is SNAP25.

3. The isolated antibody of claim 2, wherein the antibody specifically binds an alpha-exosite of a Botulinum neurotoxin A (BoNT/A) light chain (Lc) and inhibits cleavage of SNAP25 by the BoNT/A.

4. An isolated antibody that specifically binds an epitope of a Botulinum neurotoxin that is specifically bound by an antibody comprising a V_{H} comprising a CDR1, CDR2 and CDR3, wherein the CDR1, CDR2 and CDR3 are independently selected from a CDR1, CDR2 and CDR3 of a V_{H} of an antibody selected from the group consisting of Aa1, A26, A3, A16, A23, A10, Aa12, Aa6, Aa9, A8, A21, A19, Aa8, Aa5, Aa11, A8.1a, B01, B04, B12, B22, Bc1, Bc2, Bc3, Bc4, Bc5, Bc6, Bc7, Bc8, Bc9, Bc10, Bc11, Bc12, Bc13, or Bc14.

5. The isolated antibody of claim 4, wherein said antibody binds to an α-exosite of BoNT/A Lc domain

6. The isolated antibody of claim 5, wherein the antibody comprises a V_{H} CDR1 of Aa1.

7. The isolated antibody of claim 6, wherein said antibody competes for binding to a Botulinum neurotoxin with an antibody comprising:
a) a V_{H} CDR1 of Aa1;
b) a V_{H} CDR2 of Aa1; and
c) a V_{H} CDR3 of Aa1.

8. A composition comprising:
a pharmaceutically acceptable carrier; and
an isolated antibody of claim 1, wherein said antibody binds botulinum neurotoxin serotype A or B.

9. A method of treating a subject exposed to a botulinum neurotoxin, the method comprising:
administering to a subject an effective amount of an antibody of claim 1;
wherein said administering provides for inhibition of activity of botulinum neurotoxin in the subject.

10. The method of claim 9, wherein the subject suffers from intoxication by botulinum neurotoxin, and said administering is effective to reverse paralysis in the subject.

11. An isolated nucleic acid comprising a nucleotide sequence encoding an amino acid sequence of:
a V_{H} comprising a CDR1, CDR2 and CDR3 of an antibody selected from the group consisting of Aa1, A26, A3, A16, A23, A10, Aa12, Aa6, Aa9, A8, A21, A19, Aa8, Aa5, Aa11, A8.1a, B01, B04, B12, B22, Bc1, Bc2, Bc3, Bc4, Bc5, Bc6, Bc7, Bc8, Bc9, Bc10, Bc11, Bc12, Bc13, or Bc14.

12. A recombinant host cell containing the nucleic acid of claim 11.

13. A kit comprising a composition of claim 1.


SEQUENCE LISTING (87 sequences)

SEQ ID NO: 1 - protein, 137 aa, Artificial Sequence
  Synthetic Polypeptide
AVQLVDSGGGTLQAGKSLRLSCAISGLAFDGGAMGSEHRLTAGAMGWFRQAPGKDREFVAAISPRTDETYYAESLEGRFSVSRDAAATMVFLEADNVRLDDTASYYCAADEDVTPRVMGVIPHADHWGQGTLVTVSS

SEQ ID NO: 2 - protein, 137 aa, Artificial Sequence
  Synthetic Polypeptide
AVQLVESGGGAVSPGGSLKLSCTASGLAFNGGAMGSEHRLTAGAMGWFRQAPGKDREFVAAIHPRTNDTYYSGTLEDRFTISIDDAKMTVYLQADSLRLEDTAVYSCAADEDVTPRGMGVIPYAEYWGQGTQVTVSS

SEQ ID NO: 3 - protein, 128 aa, Artificial Sequence
  Synthetic Polypeptide
QVQLVESGGGLVQPGGSTNVSCEVFGGDFVSYPMAWFRQRVGIDREREFVATITPSGAGTYYADFAKGHFVVSRDNSRSTVLLQLRNLVEQDTATYFCAADDPLVGRGWDGAEGYDYWGQGTLVTVSS

SEQ ID NO: 4 - protein, 124 aa, Artificial Sequence
  Synthetic Polypeptide
AVQLVESGGGLVASGGSLRLSCAASDFKFSDYWMHWIRQAPGKEREFVASIGWNGEITDYEESIEGRFAISRDNNKYTLYLEMRDLIPEDTGLYYCAASSDYRWSRQPFEFENWGQGTQVTVSS

SEQ ID NO: 5 - protein, 122 aa, Artificial Sequence
  Synthetic Polypeptide
QVQLVESGGGMVQPGGSLRLSCKGSGSVFMFYDMGWYRQAPGNQRELVAVMGPGGNTNYADAVKGRFTVARNIDEDTVSLLMTNLEPEDTAVYYCAADFDTPWGASGRYDYWGQGTQVTVSS

SEQ ID NO: 6 - protein, 125 aa, Artificial Sequence
  Synthetic Polypeptide
EVQLVESGGGLVQPGGSLRLSCAASGFTFSGSAMSWVRQTPGKGLEWVSFISRSGSVTEYADSVKGRFTIARDNAPNTVYLQMNSLKPEDTAVYYCTADSYVDYEDDRLKWHHSWGQGTQVTVSS

SEQ ID NO: 7 - protein, 131 aa, Artificial Sequence
  Synthetic Polypeptide
DVQLVESGGGLVQPGGSLRLSCVVSGLTSTEYAIGWFRQAPGKEREGVACLVVSGHNSGRTHYSAAVKGRFTVSQDDATNTAILHMDNLQPKDTAVYYCAAEVSSGQPAVTTFWEDMYDYWGQGTQVTVSS

SEQ ID NO: 8 - protein, 116 aa, Artificial Sequence
  Synthetic Polypeptide
QVQLVESGGGLVQAGGSLRLSCAASGRTFSSYAMGWFRQAPGKEREFVAAISWSGGSTYYADSVKGRFTISRDDARNTVTLLMNSLKPEDTAVYFCNAWSLEEQYWGQGTQVTVSS

SEQ ID NO: 9 - protein, 122 aa, Artificial Sequence
  Synthetic Polypeptide
QVQLVESGGGVVQAGGSLRLSCVASGFTIDDYPVAWFRQAPGKEREAVSCIGNRGGTTYYADSVKGRFSISRDDAKDTVYLQMNNLKSEDTAVYYCVKDLGSVGPGAEYDYWGQGTLVTVSS

SEQ ID NO: 10 - protein, 127 aa, Artificial Sequence
  Synthetic Polypeptide
EVQLVESGGGLVQAGGTLRLSCAASGVRFDDSAVGWFRQAPGKESEGVSCLDSADSFTFYTDSVKGRFVTSADNAKNMIFLQMNSLKPVDTGVYYCVVYRRRHRCSAFGIANEYDYRGQGTQVTVSS

SEQ ID NO: 11 - protein, 121 aa, Artificial Sequence
  Synthetic Polypeptide
AVQLVESGGGLVQAGDSLRLSCTLSGGTFRHYAMSWFRQAPGKERVFVARIDYDGAMKYYADSVKGRFTISRDNAKNTMYLQMNSLKPEDTAVYYCNAHWDYGLGPEWDYWGQGTLVTVSS

SEQ ID NO: 12 - protein, 118 aa, Artificial Sequence
  Synthetic Polypeptide
DVQLVESGGGMVQTGGSLRLSCVVSGRTDDNYAMGWFRQAPGKEREFVAAISWSGDHTYYADSLKGRFAISRENAKNMMYLQMNSLKPEDTGVYYCNAVSTDWTTDYWGQGTLVTVSS

SEQ ID NO: 13 - protein, 125 aa, Artificial Sequence
  Synthetic Polypeptide
EVQLVESGGGLVQAGGSLRLSCTTSEGVFSNRPLGWFRQAPGKEREFVAAISWSGGYTYYGDSVKGRFTISRDAAKNTGYLQMNSLKPEDTAIYYCAGDDGEYVIPSDQNEYEFWGQGTLVTVSS

SEQ ID NO: 14 - protein, 125 aa, Artificial Sequence
  Synthetic Polypeptide
AVQLVESGGTLVQPGESLRLSCAASGFDFSSYAVGWFRQAPGKGREFVAAISWSGGYTYYGDSVKGRFTISRDAAKNTGYLQMNSLKPEDTAIYYCAGDDGEYVIPSDQNEYEFWGQGTQVTVSS

SEQ ID NO: 15 - protein, 123 aa, Artificial Sequence
  Synthetic Polypeptide
QVQLVESGGGLVQPGGSLTLVCATSGFEFGDYAIGWFRQAPGKEREGVAAVAMDDGQVYYEDSLEGRFTITSDNATKTVALLMSDLKPEDSAMYYCAADEDFLPSFVSYFDYWGQGTQVTVSS

SEQ ID NO: 16 - protein, 127 aa, Artificial Sequence
  Synthetic Polypeptide
AVQLVDSGGGLVQAGGTLRLSCAASGVRFDDSAVGWFRQAPGKESEGVSCLDSADSFTFYTDSVKGRFVISADNAKNMIFLQMNSLKPVDTGVYYCVVYRRRHRCSAFGIANEYDYRGRGTLVTVSS

SEQ ID NO: 17 - protein, 122 aa, Artificial Sequence
  Synthetic Polypeptide
DVQLVESGGGLVQPGGSLRLSCEASGFAFGTRTMNWYRQAPGKGLEWISTINHSGGFTSYADSVQDRFTISRDNAKNTVHLQMNSLNTEDTATYYCNADARITLYSGFRDYWGQGTQVTVSS

SEQ ID NO: 18 - protein, 128 aa, Artificial Sequence
  Synthetic Polypeptide
AVQLVESGGGLVQPGGSTNVSCEVFGGDFISYPMAWFRQRLGLDRERIFVATITPSGAGTYYSDFAKGRFVISRDNSRSTVLLQLRTVVEQDTATYFCAADDPLVGRGWDGAEGYDYWGQGTLVTVSS

SEQ ID NO: 19 - protein, 128 aa, Artificial Sequence
  Synthetic Polypeptide
QVQLVESGGGLVQPGQSLRLSCEVSGLILNYYSLGWFRQAQDKAREDISCIDLSQNTTLSSESLKGRFAVSRDAARNTVYLDITALEEADTATYYCATDLYPCDRILILGRDGHLEHWGQGTLVTVSS

SEQ ID NO: 20 - protein, 120 aa, Artificial Sequence
  Synthetic Polypeptide
EVQLQQSGPELVKPGASVKISCKASGYEFSRSWMNWVKQRPGKGLEWIGRIYPGDGDTNYNGKFKDKATLTADKSSSTAYMQLSSLTSEDSAVYFCARSEGFYHNLGAYWGQGTLVTVSS

SEQ ID NO: 21 - protein, 111 aa, Artificial Sequence
  Synthetic Polypeptide
QVQLVESGGNLVQPGGSLRLSCIASDSISGFNYVAWFRQSPEQARIVVAMMDAEGATNYDTSVKGRFTISKDNAKNTIYLQMNNLRPEDTALYYCNVPGVWGRGTQVTVSS

SEQ ID NO: 22 - protein, 125 aa, Artificial Sequence
  Synthetic Polypeptide
QVQLVESGGGLVQAGDSLRLSCAASGGTLGNFAMGWFRQTPGKERECVAAINWTGSSTYYSDSVRDRFTISRDNARNTMYLQMNRLKPEDTAVYYCARHQNVFGFGIRTYDYDPWGQGTQVTVSS

SEQ ID NO: 23 - protein, 111 aa, Artificial Sequence
  Synthetic Polypeptide
QVQLVESVGGVVHPGGSLRLSCVDPADSRTVAPLAWYRQAPGKQRERVARISGVGTTYYADSVKGRFTVSRDNAKNTVYLQMNSLKPEDTATYYCNRGVIWGRGTQVTVSS

SEQ ID NO: 24 - protein, 117 aa, Artificial Sequence
  Synthetic Polypeptide
QVQLVESGGGLVQPGDSLRLSCAASGETWTIADIGWWREAPGKERELVALIENSGRYTYSDAVKGRFFISRGRAQNMVYLQMNNLKPDDTAVYYCYYEDGVYDTDHWGQGTLVTVSS

SEQ ID NO: 25 - protein, 117 aa, Artificial Sequence
  Synthetic Polypeptide
DVQLVESVGGLVQPGGSLRLSCQISGDVLSINFMGWFRQYPGKQRESVARFTAEGSTKYDEDSVKGRFTISRNNAKTAMYLQMNNLKVEDTAVYYCNAIDNKWRAFYGQGTQVTVSS

SEQ ID NO: 26 - protein, 113 aa, Artificial Sequence
  Synthetic Polypeptide
QVQLVESGGGLVQAGGSLRLSCAVSRNTFSFDAIGWYRQAPGKQRELVAVFNAGESAKYLDSVKGRFTISRDRSNVYLQMNSLKPDDMDVYYCYARDYAREYRGQGTQVTVSS

SEQ ID NO: 27 - protein, 123 aa, Artificial Sequence
  Synthetic Polypeptide
DVQLVESGGGRVQAGGSLRLSCAASGFAFDDYGIGWFRQAPGKERERVSCIRSDGVTKYADSVKVRFTVSSDKTKNMVYLQMNSLKPEDSAVYYCAVRDYSKPYVSEYEYDYYGQGTLVTVSS

SEQ ID NO: 28 - protein, 118 aa, Artificial Sequence
  Synthetic Polypeptide
QVQLVESGGGLVQPGGSLRLSCAASGSISSFKSMGWYRQAPGNERDMVALITTGGDTSYSDSAKGRFTISRDSAKNTVYLQMNSLKPEDTAVYYCYARRWNFGYREMWGQGTLVTVSS

SEQ ID NO: 29 - protein, 121 aa, Artificial Sequence
  Synthetic Polypeptide
QVQLVESGGGLVQPGGSLRLSCATSDRNFKYYAMGWYRRPVGKEREFVAAIHYSGERQRYSDSVKGRFTISRDNDKNMMWLQMTDLRPEDTALYTCAAVLDYTDDTTGDYWGQGTLVTVSS

SEQ ID NO: 30 - protein, 125 aa, Artificial Sequence
  Synthetic Polypeptide
AVQLVESGGGLVRPGGSLRLSCAASASIFSVNGVGWYRRAPGKERELVAAILRDGKTNYADSVKGRFTISRDNAKDTVYLQMNSLKPEDTAVYICATGPEVTTSTMTDTSKYDYWGQGTLVTVSS

SEQ ID NO: 31 - protein, 116 aa, Artificial Sequence
  Synthetic Polypeptide
AVQLVESGGGLVQPGGSLRLSCSASGSIFSIDTMGWYRQAPGQSRLVARIARSGSSRLADPVKSRWTISRDDAKNTVYLQMDSLQPEDTGVYYCNAVSTDWTTDYWGQGTLVTVSS

SEQ ID NO: 32 - protein, 121 aa, Artificial Sequence
  Synthetic Polypeptide
AVQLVESGGGLVQAGGSLRLTCAASHGTFSKYTMGWFRQAPGKEREFVVAISWNGYTTRYGDSAKGRFTISRDNAKNTVYLQMNSLKPEDTAVYYCAARSGAGTAPPYDYWGQGTQVTVSS

SEQ ID NO: 33 - protein, 118 aa, Artificial Sequence
  Synthetic Polypeptide
QVQLVESGGGLVQAGGSLRLSCAASEDTVCISDMAWFRQDPDKAREVVARITISGSTNYADSVKGRFTISRADAKNTIYLEMSSLKPEDTAVYYCYAENVRPHEYDYWGQGTQVTVSS

SEQ ID NO: 34 - protein, 121 aa, Artificial Sequence
  Synthetic Polypeptide
QVKLVESGGGVVQPGGSLKLSCSASLFTGTFETYTMSWIRQGPGKDLEWVATVYSGGDATAYADSVKGRFIVSRDRAKNALYLQMDNLQAEDTAIYYCVMGIYSHNWPRPRSQGTLVTVSS

SEQ ID NO: 35 - protein, 19 aa, Artificial Sequence
  Synthetic Polypeptide
EVSSGQPAVTTFWEDMYDY

SEQ ID NO: 36 - protein, 18 aa, Artificial Sequence
  Synthetic Polypeptide
YRRRHRCSAFGIANEYDY

SEQ ID NO: 37 - protein, 17 aa, Artificial Sequence
  Synthetic Polypeptide
DDPLVGRGWDGAEGYDY

SEQ ID NO: 38 - protein, 17 aa, Artificial Sequence
  Synthetic Polypeptide
DEDVTPRVMGVIPHADH

SEQ ID NO: 39 - protein, 17 aa, Artificial Sequence
  Synthetic Polypeptide
DEDVTPRGMGVIPYAEY

SEQ ID NO: 40 - protein, 16 aa, Artificial Sequence
  Synthetic Polypeptide
DDGEYVIPSDQNEYEF

SEQ ID NO: 41 - protein, 16 aa, Artificial Sequence
  Synthetic Polypeptide
DDGEYVIPSDQNEYEF

SEQ ID NO: 42 - protein, 15 aa, Artificial Sequence
  Synthetic Polypeptide
SSDYRWSRQPFEFEN

SEQ ID NO: 43 - protein, 14 aa, Artificial Sequence
  Synthetic Polypeptide
DFDTPWGASGRYDY

SEQ ID NO: 44 - protein, 14 aa, Artificial Sequence
  Synthetic Polypeptide
DEDLLPSFVSDFDY

SEQ ID NO: 45 - protein, 13 aa, Artificial Sequence
  Synthetic Polypeptide
DLGSVGPGAEYDY

SEQ ID NO: 46 - protein, 12 aa, Artificial Sequence
  Synthetic Polypeptide
DSYVDYEDDRLK

SEQ ID NO: 47 - protein, 9 aa, Artificial Sequence
  Synthetic Polypeptide
HWDYGLGPE

SEQ ID NO: 48 - protein, 9 aa, Artificial Sequence
  Synthetic Polypeptide
VSTDWTTDY

SEQ ID NO: 49 - protein, 7 aa, Artificial Sequence
  Synthetic Polypeptide
WSLEEQY

SEQ ID NO: 50 - DNA, 23 bp, Artificial Sequence
  Synthetic Primer
gtcctggctgctcttctacaagg

SEQ ID NO: 51 - DNA, 21 bp, Artificial Sequence
  Synthetic Primer
atggagaggacgtccttgggt

SEQ ID NO: 52 - DNA, 55 bp, Artificial Sequence
  Synthetic Primer
cctgactatgcagctagcggtgccatggcacaggtgcagctcgtggagtctgggg

SEQ ID NO: 53 - DNA, 55 bp, Artificial Sequence
  Synthetic Primer
cctgactatgcagctagcggtgccatggcacaggtacagctggtggagtctgggg

SEQ ID NO: 54 - DNA, 55 bp, Artificial Sequence
  Synthetic Primer
cctgactatgcagctagcggtgccatggcagaggtgcagctggtggagtctgggg

SEQ ID NO: 55 - DNA, 55 bp, Artificial Sequence
  Synthetic Primer
cctgactatgcagctagcggtgccatggcagatgtgcagctggtggagtctgggg

SEQ ID NO: 56 - DNA, 55 bp, Artificial Sequence
  Synthetic Primer
cctgactatgcagctagcggtgccatggcagcggtgcagctggtggagtctgggg

SEQ ID NO: 57 - DNA, 55 bp, Artificial Sequence
  Synthetic Primer
cctgactatgcagctagcggtgccatggcagccgtgcagctggtggattctgggg

SEQ ID NO: 58 - DNA, 55 bp, Artificial Sequence
  Synthetic Primer
cctgactatgcagctagcggtgccatggcacaggtgcagctggtggagtctgtgg

SEQ ID NO: 59 - DNA, 54 bp, Artificial Sequence
  Synthetic Primer
ctgactatgcagctagcggtgccatggcacaggtaaagctggaggagtctgggg

SEQ ID NO: 60 - DNA, 57 bp, Artificial Sequence
  Synthetic Primer
cttaccttcgaagggcccgcctgcggccgctgatgagacagtgaccagggtsccctg

SEQ ID NO: 61 - DNA, 57 bp, Artificial Sequence
  Synthetic Primer
cttaccttcgaagggcccgcctgcggccgctgaggagacggtgacctgggtcccctg

SEQ ID NO: 62 - protein, 126 aa, Artificial Sequence
  Synthetic Polypeptide
QVQLVESGGGSVQAGGSLRLSCAASGYTVSTYCMGWFRQAPGKEREGVATILGGSTYYGDSVKGRFTISQDNAKNTVYLQMNSLKPEDTAIYYCAGSTVASTGWCSRLRPYDYHYRGQGTQVTVSS

SEQ ID NO: 63 - protein, 127 aa, Artificial Sequence
  Synthetic Polypeptide
XVQLQESGGGLVQAGDSLKLSCEASGDSIGTYVIGWFRQAPGKERIYLATIGRNLVGPSDFYTRYADSVKGRFAVSRDNAKNTVNLQMNSLKPEDTAVYYCAAKTTTWGGNDPNNWNYWGQGTQVTV

SEQ ID NO: 64 - protein, 129 aa, Artificial Sequence
  Synthetic Polypeptide
QVQLQESGGGLVQAGDSLKLSCEASGDSIGTYVIGWFRQAPGKERIYLATIGRNLVGPSDFYTRYADSVKGRFAVSRDNAKNTVNLQMNSLKPEDTAVYYCAAKTTTWGGNDPNNWNYWGQGTQVTVSS

SEQ ID NO: 65 - protein, 120 aa, Artificial Sequence [sequence omitted]

SEQ ID NO: 66 - protein, 127 aa, Artificial Sequence
  Synthetic Polypeptide
QVQLVASGGGSVQAGGSLRLSCAASGYTFSSYPMGWYRQAPGKECELSARIFSDGSANYADSVKGRFTISRDNAANTAYLQMDSLKPEDTAVYYCAAGPGSGKLVVAGRTCYGPNYWGQGTQVTVSS

SEQ ID NO: 67 - protein, 121 aa, Artificial Sequence
  Synthetic Polypeptide
QVQLVESGGGTVPAGGSLRLSCAASGNTLCTYDMSWYRRAPGKGRDFVSGIDNDGTTTYVDSVAGRFTISQGNAKNTAYLQMDSLKPDDTAMYYCKPSLRYGLPGCPIIPWGQGTQVTVSS

SEQ ID NO: 68 - protein, 121 aa, Artificial Sequence
  Synthetic Polypeptide
QVQLQESGGGSVQAGGSLRLSCSASGYTYISGWFRQAPGKEREGVAAIRSSDGTTYYADSVKGRFTISQDNAKNTVYLQMNSLKPEDTAMYYCAATEVAGWPLDIGIYDYWGQGTEVTVSS

SEQ ID NO: 69 - protein, 128 aa, Artificial Sequence
  Synthetic Polypeptide
QVQLQESGGGLVQAGGSLRLSCAASGRAASGHGHYGMGWFRQVPGKEREFVAAIRWSGKETWYKDSVKGRFTISRDNAKTTVYLQMNSLKGEDTAVYYCAARPVRVADISLPVGFDYWGQGTQVTVSS

SEQ ID NO: 70 - protein, 125 aa, Artificial Sequence
  Synthetic Polypeptide
DVQLVESGGGSVQAGGSLRLSCAVSGYKDRNYCMGWFRRAPGKEREGVAVIDSSGRTAYADSVKGRFTISRDVALDTAYLQMNSLKPEDTAMYYCAAGWSSLGSCGTNRNRYNYWGQGTQVTVSS

SEQ ID NO: 71 - protein, 128 aa, Artificial Sequence
  Synthetic Polypeptide
EVQLQASGGGSVQAGQSLRLSCATSGATSSSNCMGWFRQAPGKEREGVAVIDTGRGNTAYADSVQGRLTISLDNAKNTLYLQMNSLKPEDTAMYYCAADTSTWYRGYCGTNPNYFSYWGQGTQVTVSS

SEQ ID NO: 72 - protein, 127 aa, Artificial Sequence
  Synthetic Polypeptide
DVQLQESGGGLVQAGGSLRLSCAASGATGSTYDMGWFRQAPGKERESVAAINWGSAGTYYASSVRGRFTISRDNAKKTVYLQMNSLKPEDTAVYTCGAGRIGRSVFNLRRESWVTWWGQGTQVTVSS

SEQ ID NO: 73 - protein, 119 aa, Artificial Sequence
  Synthetic Polypeptide
QVQLQESGGGLVQAGGSLRLSCQASGNIFRINDMGWYRQAPGTQRELVAAITSGGSTKYADSVKGRFTISKDNAKNTVYLQMNSLKPEDTAVYYCAAEDRHRIGTVGYWGQGTQVTVSS

SEQ ID NO: 74 - protein, 125 aa, Artificial Sequence
  Synthetic Polypeptide
EVQLQESGGGLVQAGGSLRLSCAASGRTFSTYAVGWFRQAPGKEREFVGYFGTRGGRTYYADSVKGRFTIAIDNAKNTVYLQMNSLKLDDTAVYYCAVRMPYSGDYRSSGTYDYWGQGTQVTVSS

SEQ ID NO: 75 - protein, 133 aa, Artificial Sequence
  Synthetic Polypeptide
DVQLQASGGGSVQAGGSLRLSCAASGYTIGPYCMGWFRQAPGKEREGVAAINSGGGSTYYADSVKGRFTISQDNAKNTVYLLMNSLEPEDTAIYYCAADSTIYASYYECGHGLSTGGYGYDSWGQGTQVTVSS

SEQ ID NO: 76 - protein, 118 aa, Artificial Sequence [sequence omitted]

SEQ ID NO: 77 - protein, 125 aa, Artificial Sequence
  Synthetic Polypeptide
DVQLVESGGGSVQAGGSLRLSCAASGYIASINYLGWFRQAPGKEREGVAAVSPAGGTPYYADSVKGRFTVSLDNAENTVYLQMNSLKPEDTALYYCAAARQGWYIPLNSYGYNYWGQGTQVTVSS

SEQ ID NO: 78 - protein, 127 aa, Artificial Sequence
  Synthetic Polypeptide
DVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 79 - protein, 123 aa, Artificial Sequence
  Synthetic Polypeptide
QVQLQESGGGLVQAGGSLRLSCTASRRTGSNWCMGWFRQLAGKEPELVVALNFDYDMTYYADSVKGRFTVSRDSGKNTVYLQMNSLKPEDTAIYYCAARSGGFSSNRELYDGWGQGTQVTVSS

SEQ ID NO: 80 - protein, 121 aa, Artificial Sequence
  Synthetic Polypeptide
QVQLVESGGGLVQAGGSLRLSCAASGYPWTYIYMGWFRQAPGKEREGVAAMDSGGGGTLYADSVKGRFTISRDKGKNTVYLQMDSLKPEDTATYYCAAGGDALVATRYGRWGQGTQVTVSS

SEQ ID NO: 81 - protein, 123 aa, Artificial Sequence
  Synthetic Polypeptide
GSQVQLVESGGGLVQAGGSLRLSCAASGYAYTYIYMGWFRQAPGKEREGVAAMDSGGGGTLYADSVKGRFTISRDKGKNTVYLQMDSLKPEDTATYYCAAGGYELRDRTYGQWGQGTQVTVSS

SEQ ID NO: 82 - protein, 123 aa, Artificial Sequence
  Synthetic Polypeptide
GSQVQLVESGGGLVQAGGSLRLSCAASGYAYTYIYMGWFRQAPGKEREGVAAMDSGGGGTLYADSVKGRMTISRDKGKNTVYLQMDSMKPEDTATYYCAAGGYELRDRTYGQWGQGTQVTVSS

SEQ ID NO: 83 - protein, 123 aa, Artificial Sequence
  Synthetic Polypeptide
GSQVQMVESGGGLVQAGGSLRLSCAASGYAYTYIYMGWFRQAPGKEREGVAAMDSGGGGTLYADSVKGRMTISRDKGKNTVYLQMDSLKPEDTATYYCAAGGYELRDRTYGQWGQGTQVTVSS

SEQ ID NO: 84 - protein, 123 aa, Artificial Sequence; identical to SEQ ID NO: 83
  Synthetic Polypeptide

SEQ ID NO: 85 - protein, 123 aa, Artificial Sequence; identical to SEQ ID NO: 83
  Synthetic Polypeptide

SEQ ID NO: 86 - protein, 123 aa, Artificial Sequence; identical to SEQ ID NO: 83
  Synthetic Polypeptide

SEQ ID NO: 87 - protein, 123 aa, Artificial Sequence; identical to SEQ ID NO: 81
  Synthetic Polypeptide